(19) United States
(12) Patent Application Publication
(10) Pub. No.: US 2016/0015389 A1
(43) Pub. Date: Jan. 21, 2016

(54) METHODS AND SYSTEMS FOR ADVANCING AND ANCHORING SUTURE IN TISSUE

(71) Applicant: Vasostitch, Inc., Los Altos, CA (US)

(72) Inventor: Amir Belson, Los Altos, CA (US)

(21) Appl. No.: 14/801,853

(22) Filed: Jul. 17, 2015

Related U.S. Application Data
(62) Division of application No. 13/273,000, filed on Oct. 13, 2011, now abandoned.
(60) Provisional application No. 61/455,421, filed on Oct. 18, 2010.

(51) Int. Cl. A61B 17/06; A61B 19/00; A61B 17/04

(52) U.S. Cl. CPC A61B 17/06166; A61B 17/06; A61B 17/0469; A61B 19/30; A61B 19/46; A61B 2017/0409; A61B 2017/0464; A61B 2017/06052; A61B 2017/06076; A61B 2017/06009; A61B 2019/307; A61B 2019/464

(57) ABSTRACT

Suture constructs have a distal suture anchor and optionally a proximal suture anchor for mobilizing the sutures within tissue. The suture may be implanted using conventional straight, curved, or helical needles. Coupling elements may be provided in the suture constructs in order to indicate the amount of pulling force being applied to the suture when it is being manually manipulated.

### CROSS-REFERENCE APPLICATIONS

This application is a divisional of U.S. patent application Ser. No. 13/273,000 (Attorney Docket No. 39277-706.201), filed Oct. 13, 2011, which claims the benefit of U.S. Provisional Application No. 61/455,421 (Attorney Docket No. 39277-706.101), filed Oct. 18, 2010, the full disclosures of which are incorporated herein by reference. This application is also a continuation-in-part of U.S. patent application Ser. No. 13/224,666 (Attorney Docket No. 39277-703.301), filed on Sep. 2, 2011, which was a continuation of PCT/US2010/027321 (Attorney Docket No. 39277-703.601), filed on Mar. 15, 2010, which claimed the benefit of U.S. Provisional Application No. 61/210,018 (Attorney Docket No. 39277-703.101), filed on Mar. 14, 2009; and is also a continuation-in-part of U.S. patent application Ser. No. 13/169,454 (Attorney Docket No. 39277-704.201), filed on Jun. 27, 2011, which claimed the benefit of U.S. Provisional Application No. 61/398,485 (Attorney Docket No. 39277-705.101), filed on Jun. 26, 2010. The full disclosures of each of these prior applications are incorporated herein in their entirety.

### BACKGROUND OF THE INVENTION

1. Field the Invention

The present invention relates generally to devices and systems for advancing and anchoring lengths of suture in tissue. More particularly, the invention relates to anchoring suture in tissue for closing penetrations in tissue.

Sutures are very commonly used by physicians for closing wounds, incisions, fistulas, and other common tissue defects. When the defects are close to a patient's skin or other tissue surface, it is usually easy for the physician to use a needle to sew the wound closed. When the defect lies well below the skin surface, in contrast, placing sutures can be much more difficult, and a variety of tools have been developed over the years to assist in such placement. For example, numerous suturing tools have been developed for closing penetrations in the femoral artery following angioplasty and other intravascular procedures. The tools typically include a shaft which is advanced through a tissue tract which is formed through the patient's thigh to reach the femoral artery. The tools are manipulated to place the suture over the penetration, and the physician then tensions the suture to close the remote penetration through the femoral wall.

While such remote suturing tools have been very successful for femoral artery closure and other purposes (such as closing laparoscopic wounds), and have allowed procedures that were not previously possible, the use of the remote suturing tools still suffers from certain limitations. For example, in many cases it is necessary to both introduce the suture through a long tissue tract and to subsequently draw the opposite end of the tissue up through the same tract. Once the tissue is in place, it can be difficult to control the tension being placed on the suture to close the remote wound. In particular, inexperienced physicians can either supply insufficient tension, in which the wound does not fully close, or apply too much tension which can either break the suture or unnecessarily damage tissue surrounding the wound. Finally, the need to tie off the suture in the vicinity of the remote wound can also be very challenging.

For these reasons, it would be desirable to provide improved methods and systems for the advancement and anchoring of suture in tissue, particularly in procedures where remote or inaccessible wounds are being sutured. It would be particularly desirable to provide methods and tools which facilitate advancing a length of suture within solid tissue and optionally anchoring a distal end of the suture length at a remote location in the tissue. It would be further desirable to provide methods and apparatus which help the physician control the amount of tension being placed on the suture to close a wound or otherwise manipulate or reconfigure a remote tissue site. Additionally, it would be desirable if the systems and methods could also provide for anchoring a second or proximal end of the suture within the tissue to complete the wound closure or other tissue manipulation. At least of these objectives will be met by the inventions described below.

2. Description of the Background Art

Barbed sutures are described in US2010/0087855; US2008/0234731; WO1998/052473; and U.S. Pat. No. 4,964,468. Other patents and publications of interest include U.S. Pat. Nos. 7,758,595; 7,637,918; 5,545,148; 5,356,424; and 4,204,541; and U.S. Patent Publication Nos. 2009/275960; 2008/275473; 2006/253127; and 2006/212048.

### SUMMARY OF THE INVENTION

The present invention provides improved methods and systems for advancing, anchoring, and tensioning suture and tissue. While particularly useful for closing wounds, incisions, fistulas, and the like, the present invention will be useful in any procedure where a length of suture is advanced into tissue, a distal end of the suture anchored at a remote location within the tissue, and a proximal end of the suture pulled or otherwise tensioned to close a remote wound or otherwise perform a remote tissue manipulation.

In a first aspect of the present invention, a method of applying a controlled tension on tissue comprises introducing a length of suture into a tissue bed through a tissues tract. A distal end of the suture length is anchored in a distal or remote region of the tissue tract, and a pulling force is manually applied in a proximal direction on a proximal region of the suture lane to apply proximal tension on the suture and anchor. In order to control and limit tension on the remote tissue, the pulling force is applied through a coupling element which signals the physician when the pulling force exceeds a target level. The target level of force may vary widely depending on the tissue and procedure, but will typically be in the range from 2N to 25N, more typically from 2N to 15N, and often from 5N to 15N.

The physician can be signaled that the force has exceeded the target level in a variety of ways. In a first example, the coupling element can release the proximal region from the distal end of the suture, either completely or partially. Such a complete release can be achieved by providing a rupture element or “fuse” in the suture which is calibrated to break or otherwise disengage the tissue when the target level of force is met. Alternatively, a rupture region can be formed in the tissue itself, where the rupture region is selected to break or part at the desired target level of the pulling force. A partial release may be achieved by looping the suture and providing a collar or other releasable attachment means which opens or breaks when the target level of pulling force is reached.

In addition to such controlled breaking or release of the suture, the coupling element could comprise a sleeve which is attached over the proximal region of the suture. The sleeve will initially be attached to the suture by an adhesive or other mechanism which is calibrated to release the sleeve from the suture when the predetermined target level of the pulling force is reached. The physician may then manually pull on the sleeve to apply force to the suture. When the target level is reached, the sleeve will simply slide over the suture and optionally be removed.

In yet another embodiment, the coupling element may comprise a simple force measurement device which senses the pulling force being exerted on the suture. The measurement device can provide a dial, bar graph, LED array, or other visual or audible means for signaling the level of pulling force. Alternatively, the measurement device could be coupled to a visible or audible alarm which is triggered when the target level of the pulling force is met or exceeded.

The suture length is typically introduced into the tissue by advancing a needle through the tissue bed to form a tissue tract and thereafter withdrawing the needle from the tissue tract. The suture is carried by the needle, and the anchor self-deploys in the tissue as the direction of needle movement reverses from advancement to withdrawal. A particularly convenient anchor mechanism comprises barbs which are initially swept back (in a proximal direction) so that they allow the needle and suture to be advanced through the tissue but which deploy into the tissue when the needle direction is reversed and the suture is pulled in a proximal direction. In this way, the barbs may be exposed as the needle is advanced and will immediately anchor in the tissue as soon as the needle is retracted. Alternatively, however, the barbs could be confined within a passage or other receptacle within the needle as the needle is being advanced. Once the needle has reached the proper depth of advancement, the barbs can be advanced or otherwise released from the needle to anchor in the tissue before or simultaneously with proximal withdrawal of the needle.

In another aspect of the present invention, a suture construct comprises a length of suture having a distal end and proximal region. A tissue anchor is attached to the suture length near its distal end, and a coupling elements is disposed on the suture between the distal end and proximal region. The coupling elements transmits a manual pulling force from the proximal end to the distal end of the suture and signals when the pulling force exceeds a target level. The tissue anchor typically comprises barbs over at least the distal end of the suture, where the barbs are swept back in a proximal direction to allow the suture to be advanced distally through the suture but prevent the suture from being pulled proximally through the tissue as the barbs self-deploy as soon as the suture is pulled in a proximal direction. Optionally, the barbs are present only over the distal tip of the suture. In other embodiments, as described in more detail below, barbs may also be present over a proximal region of the suture, typically being oriented in the opposite direction so that the proximal end can be deployed and placed under slight tension relative to the distal end of the suture.

The coupling element may take any of the forms described above with respect to the methods of the present invention. Particularly, the coupling element may comprise a breakable link disposed between the distal and the proximal region, where the link is calibrated to break when a target level of pulling force is applied by the physician. Alternatively, the coupling element may comprise an extendable loop disposed between the distal end and the proximal region, where the loop releases from constraint on the target level of the pulling force is reached. Still further alternatively, the coupling element may comprise a breakaway sleeve placed over the proximal region of the suture, where the sleeve allows manual grasping by the user and separates from the suture when the pulling force exceeds the target level. The coupling element of the suture construct may alternatively comprise a force gauge which provides an indication or alarm when the pulling force exceeds the target level.

In a further aspect of the present invention, a method for anchoring a distal end of a length of suture in a tissue tract in a tissue bed comprises providing a needle having a tissue-penetrating distal tip with a length of suture releasably secured over or through at least a distal portion of the needle. The needle is advanced into the tissue bed so that the needle forms a tissue tract and the suture follows the tract formed in the tissue bed by the needle. Once a desired depth of needle penetration is reached, the needle advancement is reversed and the needle is retracted through the needle tract. When the direction of needle movement reverses, a distal anchor on the distal end of the suture self-deploys in the tissue bed so that the suture separates from the needle and remains in place within the needle tract after the needle is withdrawn. In this way, the needle is available a variety of tissue manipulations, wound closures, and the like.

In some embodiments, the needle may be straight and form a straight tissue tract when advanced into the tissue bed. More commonly, the needle will be curved and will formed a curved tissue tract when advanced into tissue. In still other preferred embodiments, the needle may be helical and form a helical tissue tract when advanced into tissues. In all cases, the needle will form a tissue tract which allows the needle to be advanced and retracted through the same tissue tract.

As with previous embodiments of the present invention, the anchor will typically comprise a plurality of swept back (proximally disposed) barbs over at least a distal portion of the suture. Such barbs will remain swept back while the needle and suture are being advanced and will deploy outwardly when the needle is pulled back through the tissue tract, thus preventing the suture from moving with the needle.

The barbs on the distal end of the suture may optionally be constrained while being advanced through the tissue bed, but need not be constrained. In certain embodiments, the barbs will be exposed through the needle as the needle is being advanced. As the barbs are advanced, they will immediately anchor and imbed in the tissue surrounding the tissue tract as soon as the needle advancement is reversed and the needle is withdrawn. In still other embodiments, the barbs may be constrained, for example, the present in a central passage or lumen of the needle so that they are not exposed to the tissue as the needle is being advanced. In such instances, it will be necessary to advance the barbs outside of the needle before or as the needle is being proximally withdrawn so that the suture will anchor in place.

In preferred embodiments of this method, the suture will further comprise a proximal anchor or a proximal region of the suture. The proximal anchors will also be able to self-deploy in the tissue tract and will inhibit the proximal end of the suture from moving distally. In this way, with anchors present on both the distal and proximal portions of the suture, the suture can be deployed to apply tension to and hold apposed regions of tissue together, for example, when closing a wound.

The proximal anchor will also typically comprise barbs, but the barbs will be swept distally, i.e., in an opposite direction to the barbs which are present on the distal end of the suture. The proximal barbs will thus need to be constrained as the needle and suture are being advanced. Most simply, the barbs can be confined with a lumen passage within the needle. Alternatively, the barbs may be constrained by a bio-absorbable or dissolvable material which is released over time after the suture is in place.

In a still further aspect of the present invention, a system for anchoring a distal end of a length of suture in a tissue tract in a tissue bed comprises a needle having a tissue-penetrating distal tip, typically a sharpened, chamfered, or electro-surgical tip. This system further comprises a length of suture having a self-deploying distal tissue anchor at a distal end thereof. The length of suture is releasably secured to at least a distal portion of the needle, typically being releasably secured to most of or the entire length of the needle, so that the anchor is or may become exposed to the tissue after the needle has been advanced through the tissue bed to establish the tissue tract. The distal anchor is adapted so that it becomes exposed to the tissue and anchors within the tissue as the direction of movement of the needle changes from advancement into the tissue bed to withdrawal from the tissue bed through the tissue tract. After the distal anchor has become fixed or immobilized within the tissue bed, the needle may be completely removed from the tissue tract, leaving the suture in place. It will be appreciated that such anchoring systems are particularly suitable for delivering the suture constructs described above.

In specific embodiments of the suture anchoring systems, the needle may be hollow and the length of suture may be loaded into the hollowed portion of the needle either at the time of fabrication or immediately prior to use. Alternatively, the suture may be held to the needle by a sleeve, a series of circumscribing tethers, rings, or other structures which hold the suture to the needle as it is being advanced through the tissue bed and which allow the sutured hoop be released from the needle as the needle is withdrawn from the tissue tract which has been created.

The anchors may comprise any one of a variety of structures or mechanisms which become embedded in tissue after the needle has been advanced to a desired location within the tissue bed and before or simultaneously with retraction of the needle from the tissue tract which has been created. While barbs having a plurality of swept-back tines are particularly useful, other anchor structures, such as T-tags, malecotts, expandable cages, spiral tips, and the like, may also find use. In many instances, particularly when employing barbs, at least the distal tissue anchor may be exposed ahead of or adjacent to the needle shaft as the needle is advanced. In other instances, however, the distal and other tissues anchors may be disposed within the needle lumen or be otherwise constrained during needle advancement, in which cases the anchor(s) will be deployed from or released by the needle when it is desired to anchor the suture within the tissue, such as when the needle advancement is reversed and the needle is withdrawn.

The needle may comprise any conventional geometry including straight needle bodies, curved needle bodies, helical needle bodies, and the like. The needle geometry must allow for the needle to be advanced into a bed of solid tissue to a desired depth or penetration distance and further for the needle to then be withdrawn from the tissue, leaving a tract through the tissue with the suture present in the tract. At least the straight, curved (having a constant diameter), and helical (having a constant diameter and pitch), geometries are suitable for this purpose. In preferred systems, the length of suture will further include a self-deploying proximal suture anchor which is adapted to deploy within the tissue tract to anchor a proximal suture end (in addition to the distal suture end which has already been anchored). Such proximal distal anchors may have any of the configurations described above for the distal suture anchors, preferably being a barbed structure having a plurality of tines which are swept back in the distal direction to inhibit distal movement of the proximal region of the suture after the suture has been fully deployed.

### BRIEF DESCRIPTION OF THE DRAWINGS

FIG. 1 illustrates a suture construct in accordance with the principles of the present invention.

FIGS. 2A and 2B illustrate a coupling element of the type used in the suture construct of FIG. 1, where the coupling element breaks upon application of a pulling force which exceeds the target level.

FIGS. 3A and 3B illustrate an alternative coupling element where the suture is narrowed or weakened at a location in order to provide for controlled breaking.

FIGS. 4A and 4B illustrate a breakable loop coupling element which elongates when the pulling force exceeds a target level.

FIG. 5 illustrates a coupling element which includes a bar graph pulling force indicator.

FIG. 6 illustrates a grasping sleeve knotted over a proximal region of the suture construct, where the grasping sleeve is configured to release from the suture when the pulling force exceeds target level.

FIG. 7 illustrates a suture construct having a barb-type distal suture anchor.

FIG. 8 illustrates a suture construct having both a distal barb-type tissue anchor and a proximal tissue anchor.

FIGS. 9A-9C illustrate implantation of the dual suture anchor construct of FIG. 8 into apposed tissue layers.

FIG. 10 is a perspective view of a vessel access and closure device according to the present invention shown in a partially deployed position.

FIG. 11 is an exploded view of the vessel access and closure device of FIG. 1.

FIG. 12 is cutaway view showing the internal structure of the vessel access and closure device of FIG. 10.

FIGS. 13, 14, and 15 show close up views of the distal end of the vessel access and closure device, showing how the helical suture needle with the suture on it emerges from the suturing tip and passes through the wall of the blood vessel.

FIGS. 16-31 illustrate a method of using the vessel access and closure device to open a pathway into the lumen of a blood vessel and subsequently to close the point of entry into the blood vessel.

FIG. 32 is a perspective view and FIG. 33 is a front view illustrating another embodiment of the vessel access and closure device incorporating additional features.

FIG. 34 shows an enlarged view of the helical suture needle with the suture and the suture anchor.

FIG. 35 shows an enlarged view of another variation of the suture anchor.

FIGS. 36 and 37 illustrate a helical suture needle with a toggle-shaped suture anchor.

FIG. 38 shows a suture anchor with a tissue-piercing point configured to fit into the tubular distal end of a helical suture needle.

FIG. 39 shows another suture anchor with a tissue-piercing point configured with a multiplicity of small barbs.

FIGS. 40 and 41 show a distal portion of a tubular helical suture needle with a suture anchor made of a superelastic or shape memory NiTi alloy wire.

FIGS. 42, 43, and 44 show a distal portion of a tubular helical suture needle with a suture anchor configured as an expandable cage.

FIG. 45 illustrates a system for establishing transapical access to a heart chamber constructed in accordance with the principles of the present invention and including a helical needle driver, a dilator, a straight needle, and optionally a guidewire.

FIGS. 46A and 46B illustrate the helical needle driver of FIG. 45 in detail, with the helical needle retracted in FIG. 46A and the helical needle advanced in FIG. 46B.

FIGS. 47A and 47B illustrate a barbed suture anchor and a T-bar suture anchor, respectively, emerging from distal end of a helical needle.

### DETAILED DESCRIPTION OF THE INVENTION

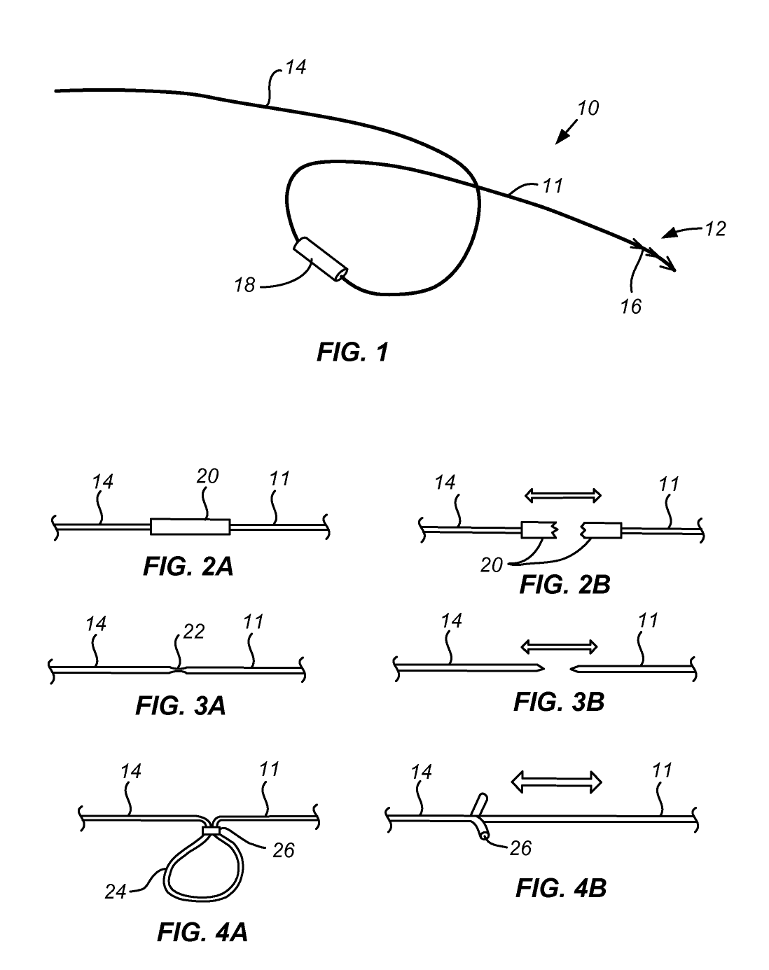

A suture construct 10 constructed in accordance with the principles of the present invention is illustrated in FIG. 1. The suture construct comprises a length of suture having a distal portion 11 with a distal tip 12 and a proximal region 14. A barb structure or other tissue anchor 16 is disposed at or near the distal tip 12, and a coupling element 18 is disposed between the distal portion 11 and proximal region 14 of the suture.

The coupling element 18 is provided to alert the user when a pre-determined target level of pulling force is being applied through the suture to the tissue anchor 16 when the suture is in tissue and the anchor immobilized at the end of a tissue tract.

In a first exemplary embodiment, the coupling element may be in the form of a breakable link or fuse 20. The link or fuse 20 will be configured so that it will remain intact (FIG. 2A) so long as the pulling force applied to the proximal suture portion 14 remains below the target level and will fracture or otherwise decouple (FIG. 2B) when the pulling force exceeds that level, pullin the distal portion 11 and proximal region 14 of the suture apart, as indicated by the arrow in FIG. 2B.

A second exemplary coupling element embodiment comprises a simple narrowed or weakened region 22 formed into the suture length between the distal portion 11 and the proximal region 14, as shown in FIG. 3A. By applying a pulling force above the target level in the direction of the arrow, the suture will break apart as shown in FIG. 3B.

The suture, however, need not be configured to break. Instead, as shown in FIGS. 4A and 4B, a loop 24 may be formed in the suture and held together by a breakable collar 26, as shown in FIG. 4A. By applying a proximal pulling force on the proximal region 14, the collar 26 will break, as shown in FIG. 4B, allowing the length of suture to increase in a manner which alerts the user.

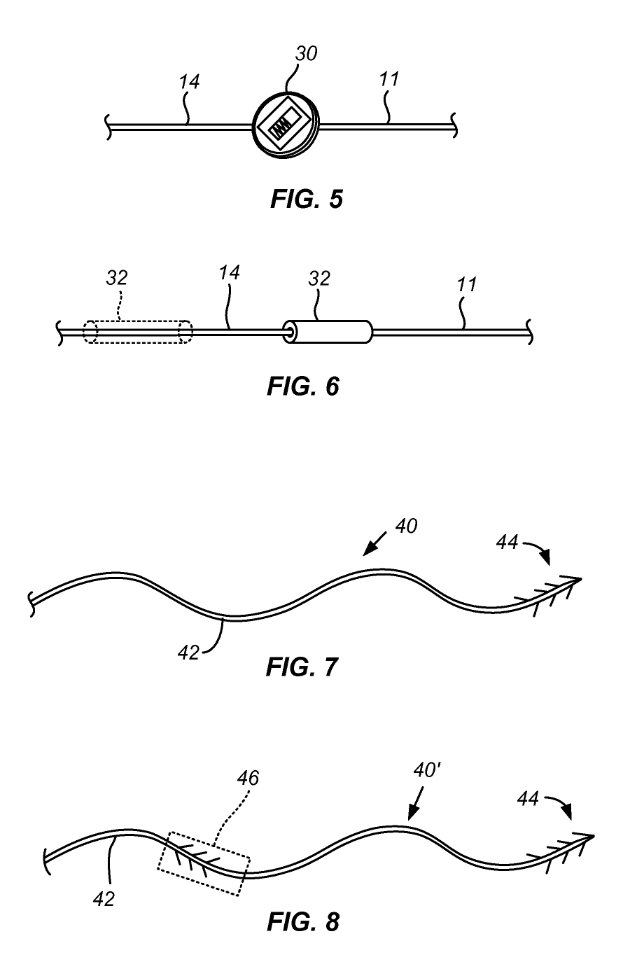

In a still further embodiment, the coupling element may be in the form of a gauge or indicator 30, as shown in FIG. 5. The particular gauge illustrated has a bar graph which can indicate the amount of tension as a portion or percentage of the target level of the pulling force. In this way, the user knows when the target level has been reached without the need for the suture to break or extend.

As a still further alternative embodiment, a breakaway sleeve 32 may be positioned on the suture as illustrated in FIG. 6. The sleeve 32 will initially be fixed to the suture at a desire location between the distal portion 11 and the proximal region 14. The user will grasp the breakaway sleeve 32 and apply the pulling force via the sleeve. When the target level of the pulling force is reached, the sleeve will break away from its attachment to the suture, thus alerting the user that sufficient force has been applied.

Referring now to FIG. 7, the methods of the present invention for anchoring a distal end of a length of suture in a tissue tract may be performed with suture constructs which do not include the coupling element as described above. Such methods, for example, may be performed with suture constructs 40 having a length of suture material 42 with an anchor 44 at their distal ends, as shown in FIG. 7. Additionally, the methods may be performed with suture constructs 40′, as shown in FIG. 8. Where a length of suture 42 not only includes the distal anchor 44 but also a proximal anchor 46 which is spaced proximally by selected distance from the distal anchor 44, typically a distance in the range from 1 cm to 10 cm. Both the distal anchor 44 and proximal anchor 46 will preferably comprise barb assemblies with a plurality of swept-back tines with the distal tines being disposed or swept back in the proximal direction and the proximal tines being disposed or swept back in the forward direction. In this way, the suture construct 40′ may be deployed in solid tissue with the distal anchor 44 and proximal anchor 46 maintaining the suture there between in tension.

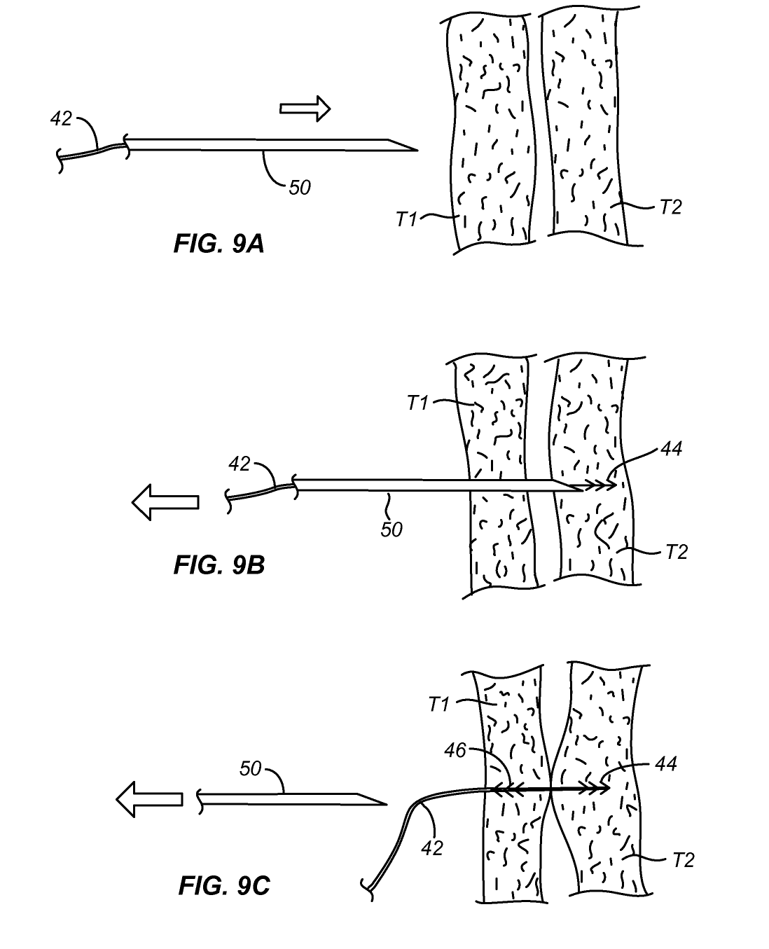

The suture construct 40′ may be disposed in tissue using a simple straight needle 50 as illustrated in FIGS. 9A-9C. The future construct 40 is disposed within a lumen of the needle such that the distal and proximal anchors are fully constrained therein, as shown in FIG. 9A. The needle is then advanced through apposed tissue layers T1 and T2 and the distal anchor 44 deployed from the tip of the needle 50 into the tissue, as shown in FIG. 9B. The tines of the distal anchor 44 deploy the tissue so that proximal movement of the distal tip is resisted, i.e., the tines of the barbed anchor 44 become embedded in the tissue. As the needle 50 is withdrawn, the distal end of the suture length 42 remains immobile within the tissue, and the suture may be tensioned in order to draw the tissue together as shown in FIG. 9C. Once the tissue is in a desired approximated configuration, the needle can be withdrawn from over the proximal anchor structure 46, anchoring both ends of the suture in place and holding the tissue in the apposed configuration illustrated. The remaining free end of the suture length 42 may then be cut off or utilized for other purposes.

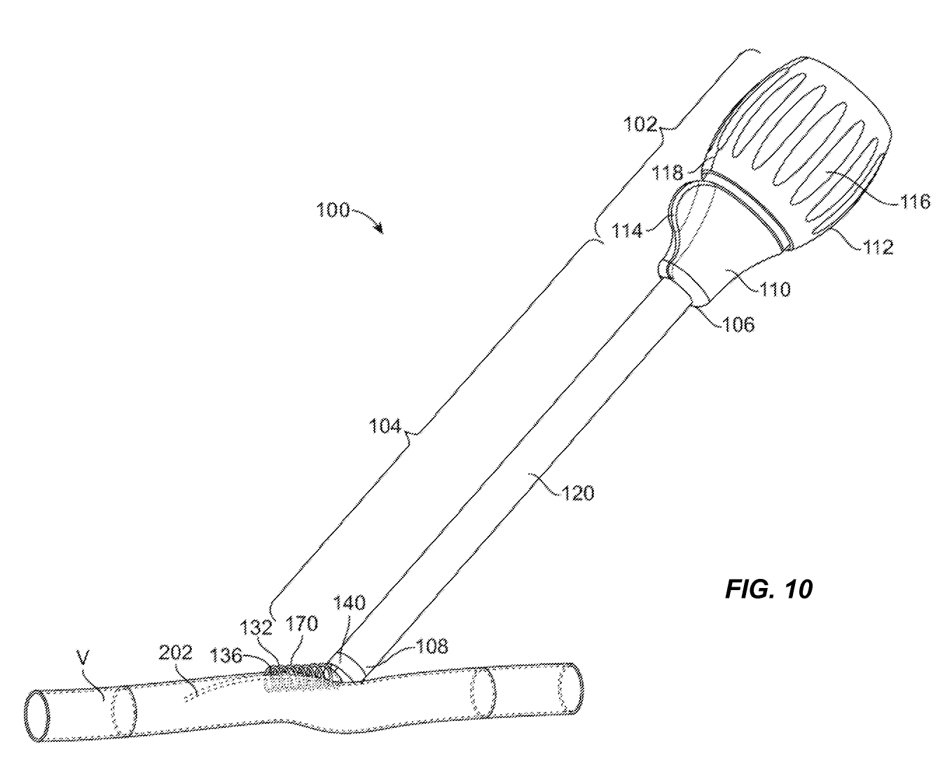

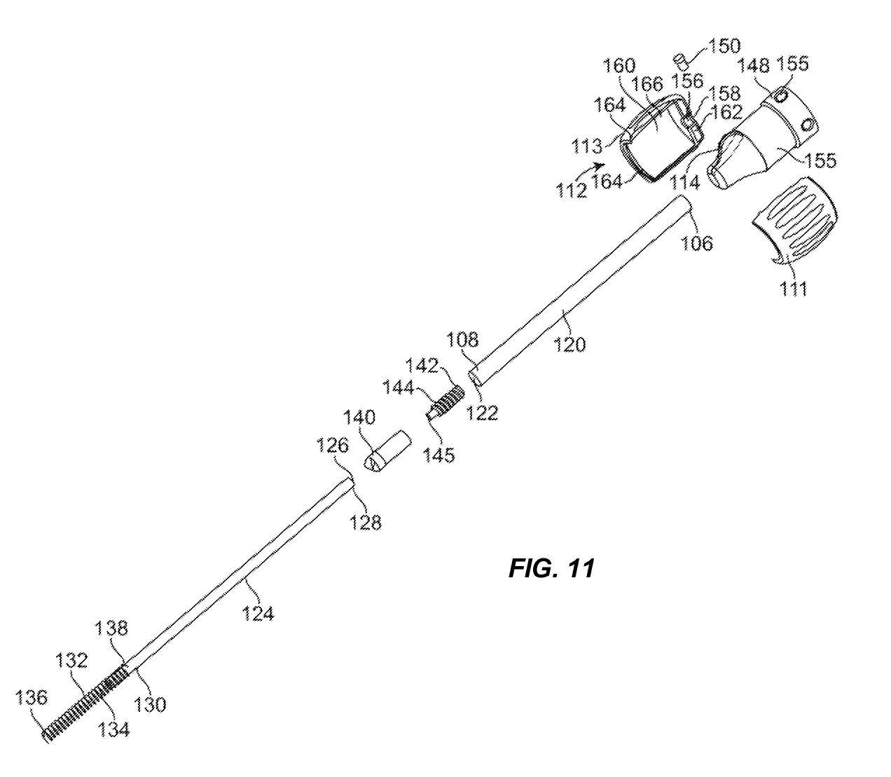

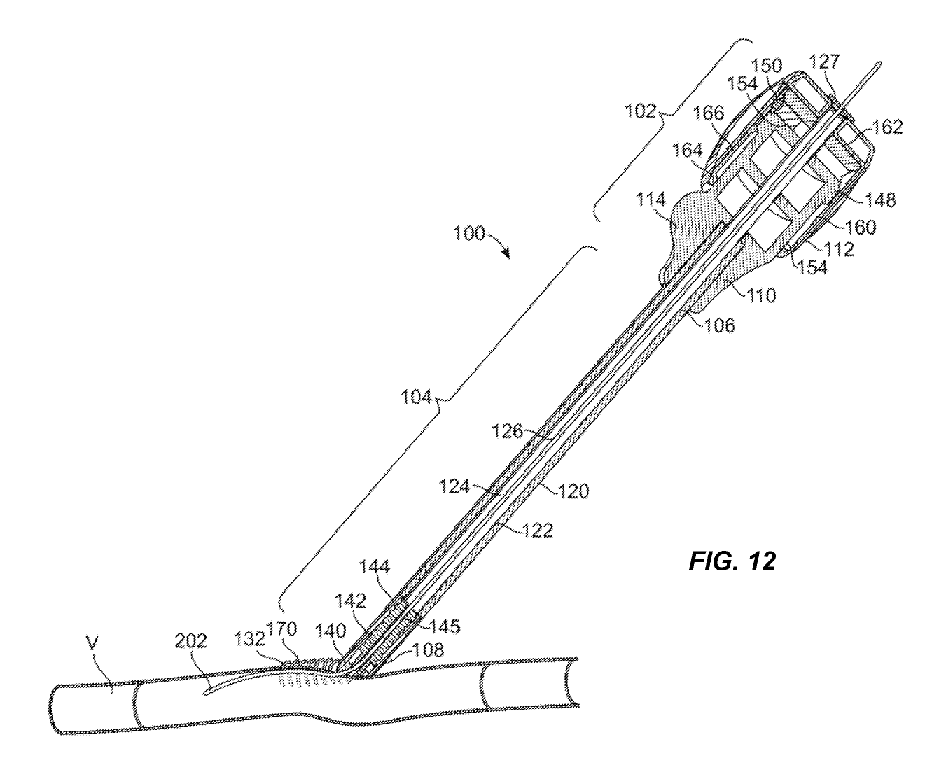

FIG. 10 is a perspective view of a vessel access and closure device 100 according to the present invention shown in a partially deployed position for placing a running suture in the wall of a blood vessel V. FIG. 11 is an exploded view of the vessel access and closure device 100 of FIG. 10. FIG. 12 is cutaway view showing the internal structure of the vessel access and closure device 100 of FIG. 10.

The vessel access and closure device 100 has an elongated shaft portion 104 with a proximal end 106 and a distal end 108. A proximal handle 102 is connected to the elongated shaft portion 104 at the proximal end 106. The proximal handle 102 has a stationary portion 110 and a rotating portion 112 located proximal to the stationary portion 110. Preferably, the rotating portion 112 of the proximal handle 102 will have a contour 116 and/or texture configured for easy gripping by the operator for applying torque to rotate the rotating portion 112. Additionally, the rotating portion 112 may have a line 118 or other marking to indicate the rotational position of the rotating portion 112. Preferably, the stationary portion 110 of the proximal handle 102 is configured with a wing-shaped raised portion 114, preferably located at a 12 o'clock position on the closure device 100, that serves as a handle to apply torque to resist rotation of the device 100 when the rotating portion 112 is rotated and as a visual and tactile indicator to the operator of the device orientation.

As shown in FIG. 11, the elongated shaft portion 104 has a hollow, tubular outer shaft 120 with an inner lumen 122 that, when assembled as in FIG. 12, is fixed at its proximal end 106 to the stationary portion 110 of the proximal handle 102. Positioned within the inner lumen 122 of the outer shaft 120 is a hollow, tubular inner shaft or torque tube 124 with a central lumen 126 that, when assembled, is fixed at its proximal end 128 to the rotating portion 112 of the proximal handle 102. Preferably, the outer shaft 120 and the torque tube 124 are each constructed of stainless steel tubing or, alternatively, another metal, such as a titanium or cobalt-chromium alloy, a rigid polymer or a reinforced polymer composite. A helical suture needle 132 having a multiplicity of helical turns or coils is connected at its proximal end 138 to the distal end 130 of the torque tube 124. The helical suture needle 132 has a central passage 134 that is axially aligned with the central lumen 126 of the torque tube 124. For ease of manufacture and assembly, the helical suture needle 132 will preferably have an outer diameter that is approximately the same as the outer diameter of the torque tube 124. The helical suture needle 132 is configured to carry a suture thread along the helical coil. For this purpose, the helical suture needle 132 may be hollow or it may be solid, but with a groove or channel to carry the suture, as will be discussed in greater detail below. Preferably, the helical suture needle 132 is constructed of a metal, such as stainless steel or a titanium, nickel-titanium or cobalt-chromium alloy. The distal end 136 of the helical suture needle 132 will typically be sharpened into a tissue-penetrating point, however other possible configurations are described below.

A specially contoured suturing tip 140 is attached at the distal end 108 of the outer shaft 120 and proximal to it, inside the inner lumen 122 of the outer shaft 120, is attached a needle guide 142 with a helical groove 144 on its exterior having approximately the same diameter and pitch as the helical suture needle 132. A guidewire lumen 145 extends through the center of the needle guide 142 and aligns with the central lumen 126 of the torque tube 124. Preferably, a hemostasis valve 127, such as an elastic membrane with a hole or slit through it, is provided at the proximal end of the handle 102 to prevent excessive bleeding through the central lumen 126. The hemostasis valve 127 provides a sliding seal for insertion of the guidewire 202 and, optionally, for the positioning member 242, dilator 210 and/or introducer sheath 222 described below. The needle guide 142 and the suturing tip 140 do not rotate with respect to the outer shaft 120. The needle guide 142 may be attached to or integral with the suturing tip 140 or it may be attached directly to the outer shaft 120. When assembled, the helical suture needle 132 rides in the helical groove 144 of the needle guide 142. Alternatively, the needle guide 142 may be made without the helical groove 144.

The stationary portion 110 of the proximal handle 102 is preferably made of a rigid polymer material, such as polycarbonate, nylon, ABS, polyurethane, etc., and may be molded as one piece or two and assembled onto the proximal end 106 of the outer shaft 120 by insert molding, compression, adhesives, pins, set screws, keys, splines or any other secure method. In the example shown, the proximal end 106 of the outer shaft 120 is inserted into a cylindrical pocket 146 in distal end of the stationary portion 110 of the proximal handle 102 and secured with adhesive. The stationary portion 110 of the proximal handle 102 has a cylindrical portion 154 and an annular boss 148 that is just slightly larger in diameter than the cylindrical portion 154. A ball detent 150 or the like is inserted into a transverse hole 155 in the annular boss 148, preferably located at a 12 o'clock position.

For ease of manufacture and assembly, the rotating portion 112 of the proximal handle 102 is preferably molded as two pieces 111, 113 and assembled onto the proximal end 128 of the torque tube 124 and the stationary portion 110 of the proximal handle 102 at the same time. The two pieces 111, 113 of the rotating portion 112 may be joined together by adhesives, screws, etc. The proximal end 128 of the torque tube 124 fits into a central bore 156 at the proximal end of the rotating portion 112 of the proximal handle 102 and is secured by an adhesive. Optionally, an annular ridge 158 may be molded at the proximal end of the central bore 156 to assure proper axial positioning of the torque tube 124 during assembly. During assembly, the line 118 on the rotating portion 112 is axially aligned with the distal end 136 of the helical suture needle 132.

The rotating portion 112 of the proximal handle 102 has an internal cylindrical portion 160 that is delineated on the proximal end by the proximal wall 162 of the rotating portion 112 of the proximal handle 102 and on the distal end by an inwardly projecting annular flange 164. The internal cylindrical portion 160 has an inner diameter that is just slightly larger than the outer diameter of the annular boss 148 on the stationary portion 110 of the proximal handle 102. The inwardly projecting annular flange 164 has an inner diameter that is just slightly larger than the outer diameter of the cylindrical portion 154 of the stationary portion 110 of the proximal handle 102, but slightly smaller than the annular boss 148. Thus, the rotating portion 112 of the proximal handle 102 is able to rotate and move axially on the stationary portion 110, but the axial movement in the proximal direction is limited by the inwardly projecting annular flange 164 and in the distal direction by the proximal wall 162 of the rotating portion 112.

A longitudinal groove 166 is molded into the internal cylindrical portion 160 of the rotating portion 112 of the proximal handle 102, preferably located at a 12 o'clock position where the two pieces 111, 113 of the rotating portion 112 join. The longitudinal groove 166 interacts with the ball detent 150 each time it rotates past the 12 o'clock position to give an audible and/or tactile indication to the operator that the rotating portion 112, and hence the distal end 136 of the helical suture needle 132 also, is rotating past the 12 o'clock position.

As the rotating portion 112 of the proximal handle 102 rotates in the direction of the helix of the helical suture needle 132 (clockwise in the example shown), the helical suture needle 132 engages the helical groove 144 on the needle guide 142, moving the helical suture needle 132, the torque tube 124 and the rotating portion 112 distally with respect to the outer shaft 120 and the stationary portion 110 of the proximal handle 102.

In an alternative configuration, the rotating portion 112 of the proximal handle 102 may be molded as a single piece that is threaded onto the stationary portion 110 of the proximal handle 102. The screw threads between the rotating portion 112 and the stationary portion 110 will preferably have a pitch that is equal to the pitch or coil-to-coil distance of the helical suture needle 132 so that the rotating portion 112 will advance and retract synchronously with the helical suture needle 132. This configuration controls the axial movement of the rotating portion 112 with respect to the stationary portion 110 and obviates the need for the annular boss 148 and the inwardly projecting annular flange 164 described above.

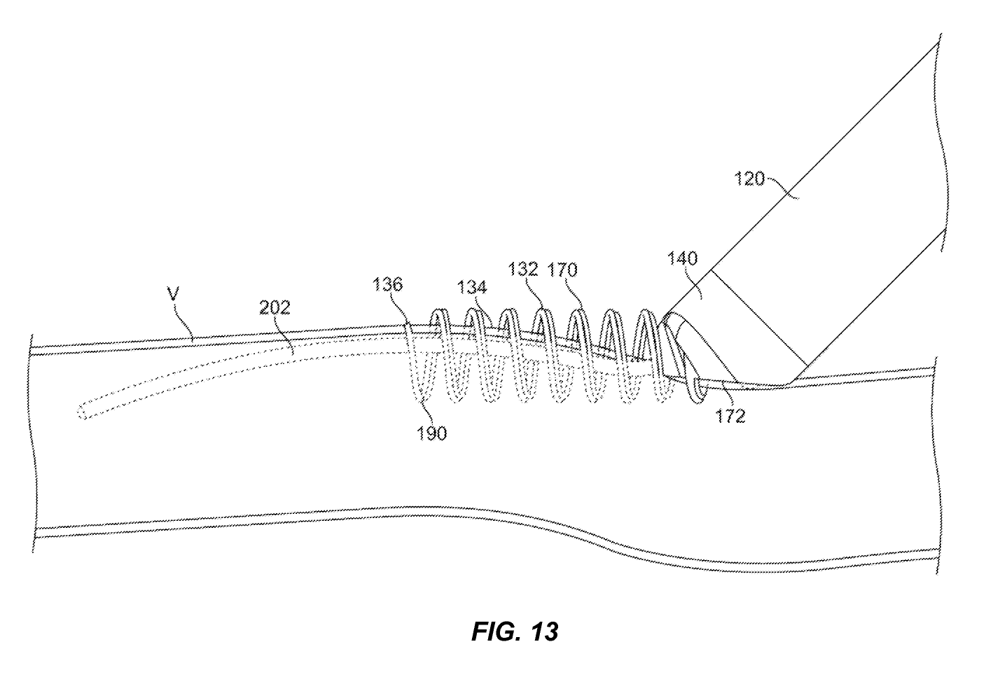

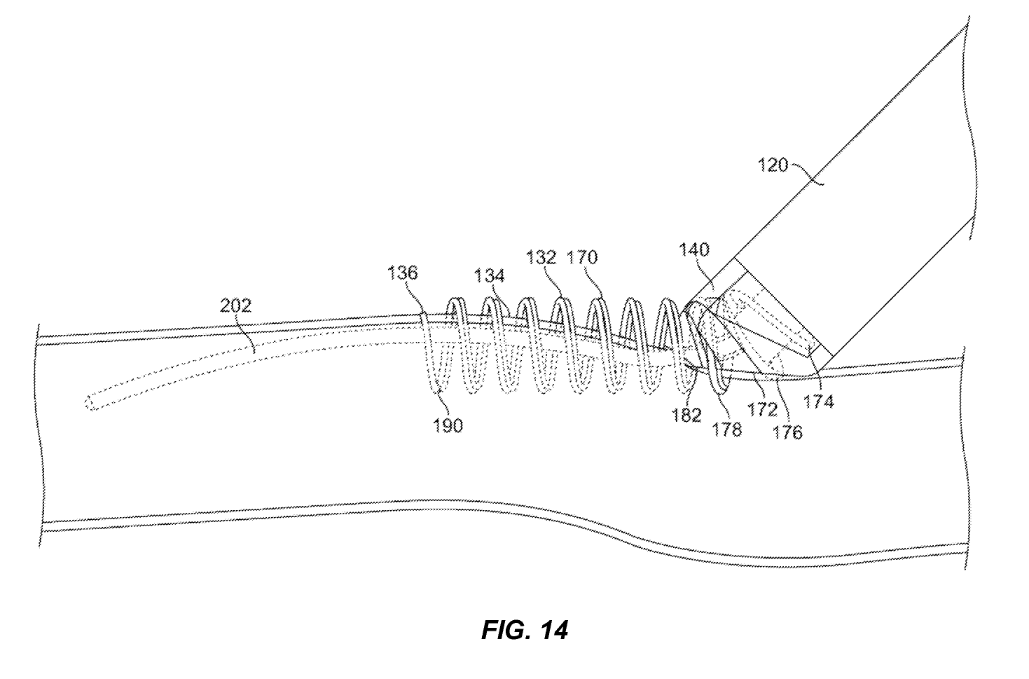

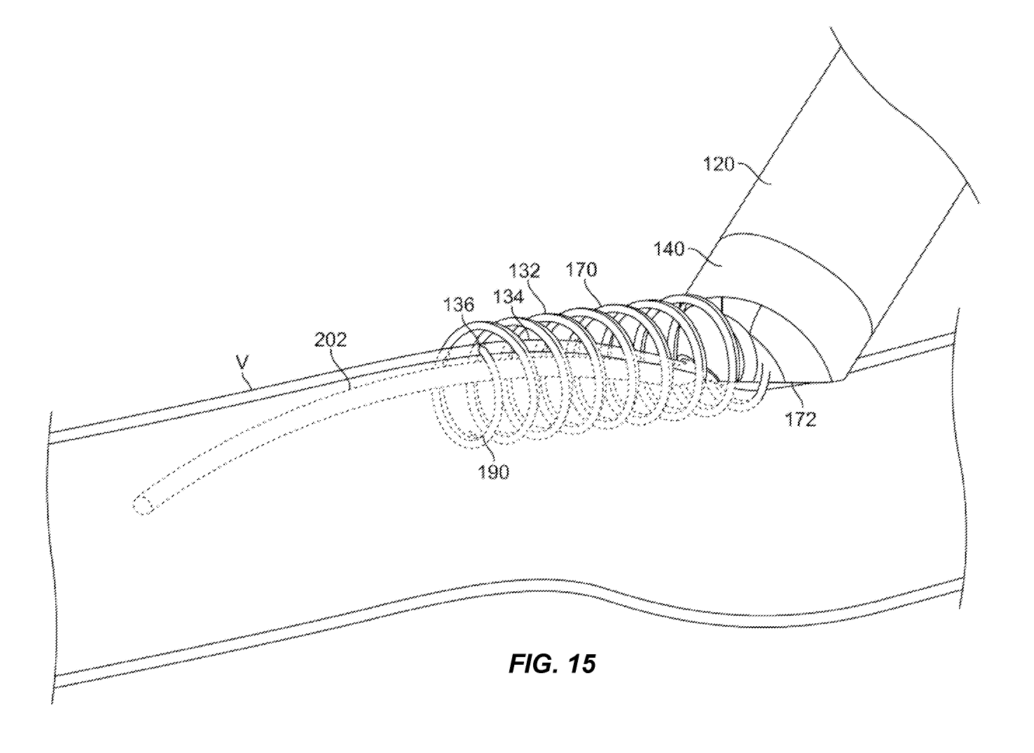

FIGS. 13, 14, and 15 show close up views of the distal end of the vessel access and closure device 100, showing how the helical suture needle 132, with the suture 170 on it, emerges from the suturing tip 140 and passes through the wall of the blood vessel V. The suturing tip 140 has a distal face 172 that is at an angle of approximately 45 degrees from the longitudinal axis of the elongated shaft portion 104. In other embodiments, this angle can be from 15 to 135 degrees. The distal face 172 of the suturing tip 140 may be flat or it may have a curvature that is a section of a cylinder with a radius of curvature approximately equal to the radius of the blood vessel V that it is intended to be used with. The suturing tip 140 is configured so that it gradually redirects the helical suture needle 132 from its orientation inside of the closure device 100 where the helical suture needle 132 is concentric with the longitudinal axis of the elongated shaft portion 104 to an orientation where the helical suture needle 132 is concentric with an axis that is at an angle of approximately 45 degrees from the longitudinal axis of the elongated shaft portion 104. FIG. 14, which shows a phantom view of the suturing tip 140, illustrates how this is accomplished. The interior of the suturing tip 140 defines a curving helical path that gradually redirects the helical suture needle 132 over a course of 2-3 turns of the helical coil. In FIG. 5, coil 174 is concentric with the longitudinal axis of the elongated shaft portion 104. Coil 176 has been skewed approximately 15-30 degrees from coil 174 and coil 178 has been skewed another approximately 15-30 degrees from coil 176. Coil 180 and the remainder of the coils distal to it are approximately concentric with an axis that is at an angle of approximately 45 degrees from the longitudinal axis of the elongated shaft portion 104. Another way to envision this geometry is that the transitional coils 174, 176, 178 are bunched up together on the inside of the curve, which causes the helical suture needle 132 to change direction by approximately 45 degrees. Another feature of the suturing tip 140 is that coils 174 and 176 are entirely inside of the suturing tip 140, whereas coil 178 is exposed along approximately one half or a turn so that the distal end 136 of the helical suture needle 132 can take a first bite of the vessel wall V for placing the suture 170 as it rotates past this position. Coil 180 and the remainder of the coils distal to it are entirely exposed for additional bites of the vessel wall V.

A guidewire lumen 182 passes through the suturing tip 140 making a gradual bend of approximately 135 degrees to emerge approximately parallel to the distal face 172 of the suturing tip 140. When the device 100 is assembled, the proximal end of the guidewire lumen 182 of the suturing tip 140 aligns with the guidewire lumen 145 of the needle guide 142 and the central lumen 126 of the torque tube 124.

Another important feature of the vessel access and closure device 100 is a suture anchor 190 that is connected to the distal end of the suture 170. Various forms of the suture anchor 190 are shown in FIGS. 25-35. Initially, the suture anchor 190 is located at or near the distal end 136 of the helical suture needle 132. The suture anchor 190 is configured so that, as the helical suture needle 132 moves through the vessel wall in the distal direction, the suture anchor 190 moves smoothly forward without catching on the tissue, however, when the direction of the helical suture needle 132 is reversed, the suture anchor 190 opens or spreads and anchors the distal end of the suture 170 to the vessel wall. The helical suture needle 132 leaves a loose helical coil of suture 170 in the vessel wall as it is withdrawn. Release of the suture 170 from the helical suture needle 132 may be passive or active.

FIGS. 16-29 illustrate a method of using the vessel access and closure device 100 to open a pathway into the lumen of a blood vessel V and subsequently to close the point of entry into the blood vessel V. This method, and variations of it, may be performed with any of the embodiments of the vessel access and closure device 100 described herein. The method is initiated using the Seldinger technique to access the lumen of the blood vessel V, which may be an artery or a vein. As shown in FIG. 16, an access needle 200 is used to puncture the patient's skin and create a tract through the tissue and into the lumen of the blood vessel V. Optionally, a skin nick may be made with a scalpel before or after inserting the access needle 200 to prevent tearing of the patient's skin later in the procedure. Preferably, the needle puncture is made at an angle of approximately 30 to 45 degrees from the central axis of the blood vessel V. Blood flashback through the access needle 200 may be used to verify that the distal tip of the access needle 200 is in the lumen of the blood vessel V and whether an artery or vein has been correctly accessed. (For clarity, the patient's skin and the tissue surrounding the blood vessel V have been left out of these illustrations.)

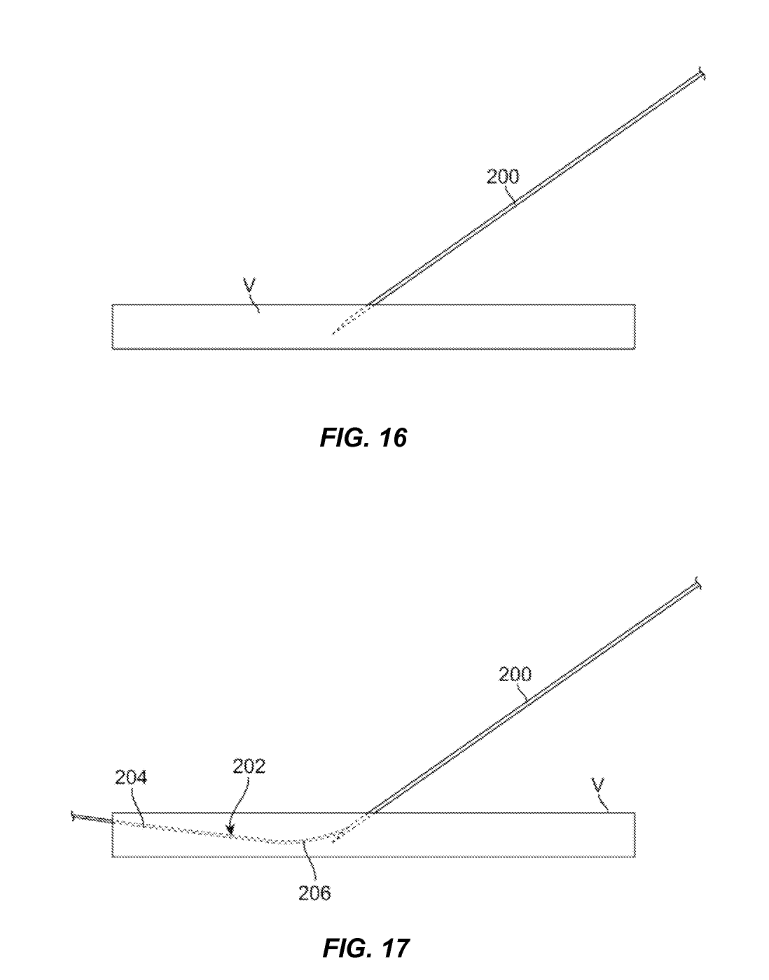

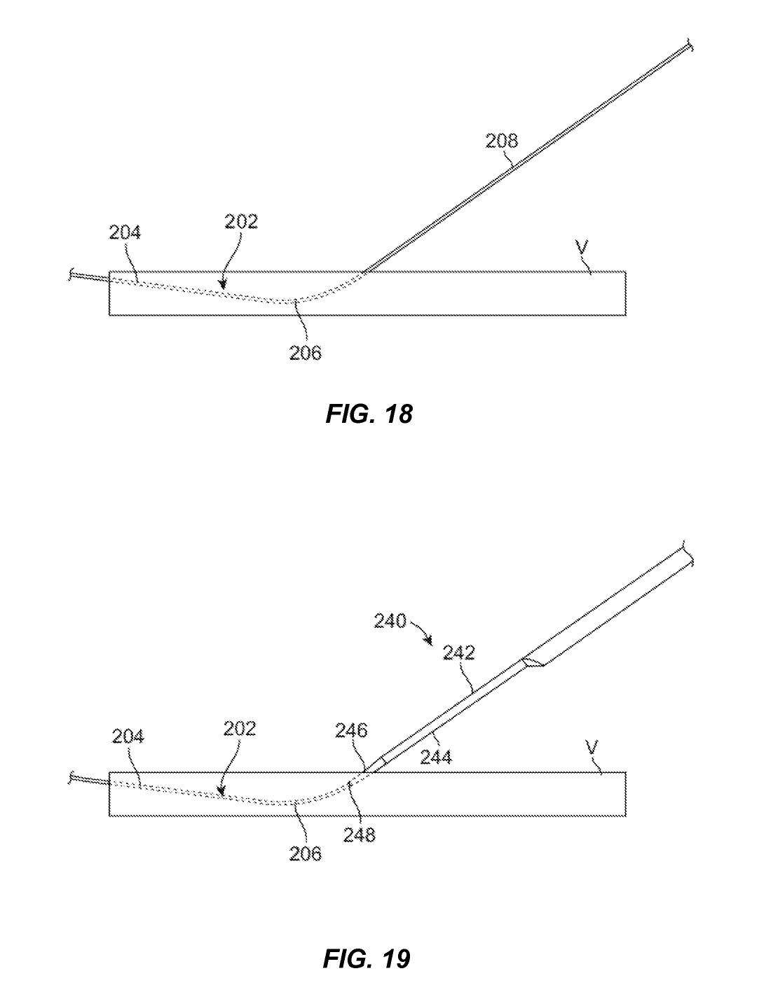

Next, a special guidewire 202 is inserted through the access needle 200 into the lumen of the blood vessel V, as shown in FIG. 17. The guidewire 202 has a bend 206 of approximately 135 degrees between a distal portion 204 and a proximal portion 208 that is used to locate the wall of the blood vessel V during subsequent steps of the method. Optionally, the guidewire 202 may have a J-shaped tip to avoid potential injury to the interior of the blood vessel, as is known in the art. The operator can feel when the bend 206 in the guidewire 202 has exited the distal tip of the access needle 200 and entered the lumen of the blood vessel V, as shown in FIG. 17. At this point the access needle 200 is withdrawn, leaving the guidewire 202 to maintain a pathway through the tissue tract created by the access needle 200 and into the lumen of the blood vessel V, as shown in FIG. 18.

Optionally, the tissue tract can be dilated using a series of tapered dilators or using an expandable dilator, such as an inflatable balloon, as is known in the art. Whether this step is necessary, depends in part on how large the tissue tract needs to be and how resistant the tissue is to passage of the shaft portion 104 of the vessel access and closure device 100. In an alternative method, a tissue cutdown can be used to access the exterior of the blood vessel V before inserting the access needle 200.

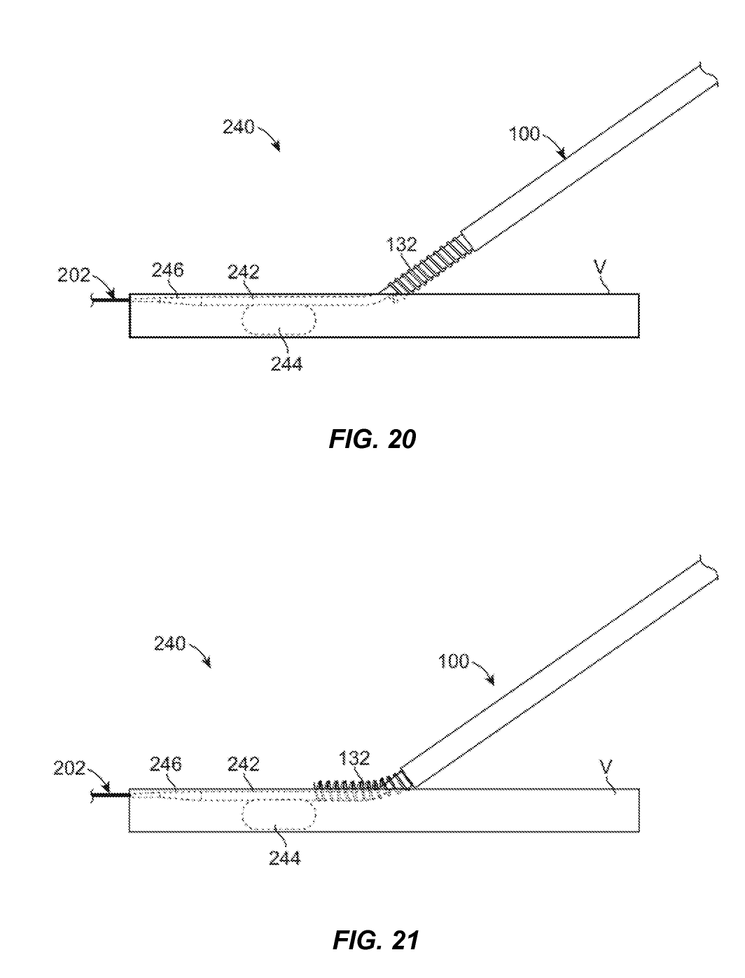

Next, the proximal portion 208 of the guidewire 202 is inserted into the guidewire lumen 182 in the suturing tip 140 and through the guidewire lumen 145 of the needle guide 142 and the central lumen 126 of the torque tube 124 to emerge from the proximal handle 102. The shaft portion 104 of the vessel access and closure device 100 is advanced through the tissue tract while pulling upward gently on the guidewire 202 to position the bend 206 of the guidewire 202 at the wall of the blood vessel V, as shown in FIG. 19. The operator will be able to feel when the shaft portion 104 of the device 100 has reached the blood vessel V and the distal face 172 of the suturing tip 140 is against the exterior of the vessel wall, as shown in FIG. 20. Proper positioning of the suturing tip 140 can be verified fluoroscopically or with ultrasound imaging.

As shown in FIGS. 19, 20 and 21, the apparatus may optionally include an additional positioning device 240 that helps to assure that the suture is placed in the near wall of the blood vessel V as intended. The positioning device 240 may be a separate device insertable through the vessel access and closure device 100 or it may be integrated into vessel access and closure device 100. The positioning device 240 has an elongated tubular guiding element 242 with a guidewire lumen 248 that is sized to fit over the guidewire 202. The guiding element 242 has a tapered dilating tip 246 at its distal end and a biasing element in the form of an inflatable balloon 244 mounted on one side of the guiding element 242. An inflation lumen connected to the balloon 244 extends through the guiding element 242 to a proximal hub (not shown) on the proximal end of the guiding element 242. Preferably, the balloon 244 has a very low deflated profile, as shown in FIG. 10, so that it can fit through the lumens 182, 145, 126 in the elongated shaft portion 104 of the vessel access and closure device 100. The balloon 244 is preferably located at a 6 o'clock position on the guiding element 242. A line or other mark (not shown) at a 12 o'clock position on the proximal end of the guiding element 242 allows the operator to properly orient the balloon 244 during insertion. The inflated profile may be cylindrical, as shown in FIG. 20, or it may be spheroidal or other shapes described herein. The balloon 244 may be made of compliant or noncompliant material. The diameter of the inflated balloon 244 is such that it biases the guiding element 242 toward the near wall of the blood vessel V, so that the helical suture needle 132 will be properly oriented with respect to the blood vessel wall when it is advanced, as shown in FIG. 21.

Optionally, the positioning device 240 may also include a needle guide 241 on the guiding element 242 proximal to the balloon 244. The needle guide 241 has a diameter that is larger than the diameter of the guiding element 242 and is eccentrically positioned on the guiding element 242, as best seen in FIG. 19. The needle guide 241 may be cylindrical or it may have an elliptical or D-shaped cross section. The needle guide 241 assures that the helical suture needle 132 will be properly aligned with the wall of the blood vessel V when it is advanced. The eccentric positioning of the needle guide 241 allows the helical suture needle 132 to take at least one, and more preferably two bites, of the blood vessel wall proximal to the puncture site, as shown in FIGS. 20 and 21.

The rotating portion 112 of the proximal handle 102 is rotated clockwise like a knob while holding the stationary portion 110 to prevent it from rotating. The torque tube 124 transfers the rotation to the helical suture needle 132 which engages the helical groove 144 on the needle guide 142 and advances distally, as shown in FIG. 21. The proximal handle 102 may include a visual indication of the position of the stationary portion 110 with respect to the rotating portion 112 and/or a counter for recording the number of turns as an indication of the position of the helical suture needle 132. As can be seen in FIG. 23, the first two stitches or bites of the vessel wall made by the helical suture needle 132 are proximal to the point where the guidewire 202 enters the vessel wall. Approximately 4 to 8 more stitches are made distal to the point where the guidewire 202 enters the vessel wall.

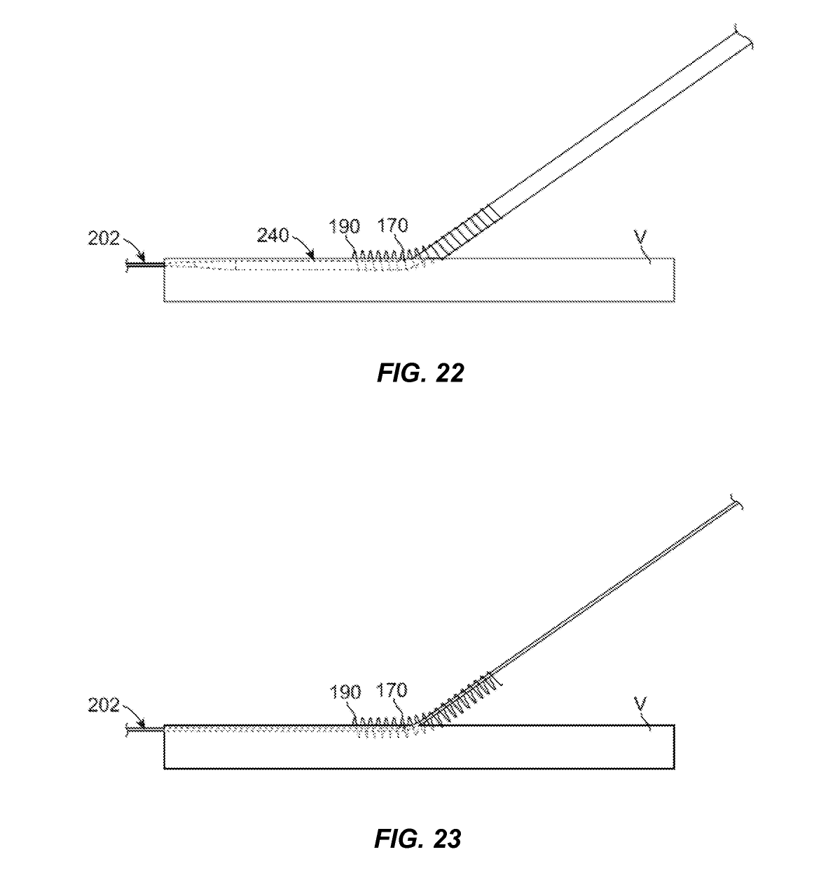

After a sufficient number of stitches have been placed, the clockwise rotation is stopped, preferably when the distal end 136 of the helical suture needle 132 and the suture anchor 190 are at approximately the 12 o'clock position outside of the blood vessel V. The rotating portion 112 of the proximal handle 102 is then rotated counterclockwise to withdraw the helical suture needle 132. The suture anchor 190 engages the vessel wall and prevents the suture 170 from backing out. A loose helical coil of suture 170 is left behind as the helical suture needle 132 withdraws, as shown in FIG. 22.

The vessel access and closure device 100 is withdrawn from the tissue tract leaving the helical coil of suture 170 in the vessel wall and the guidewire 202, which maintains a pathway through the tissue tract and through the center of the helical coil of suture 170, as shown in FIG. 23.

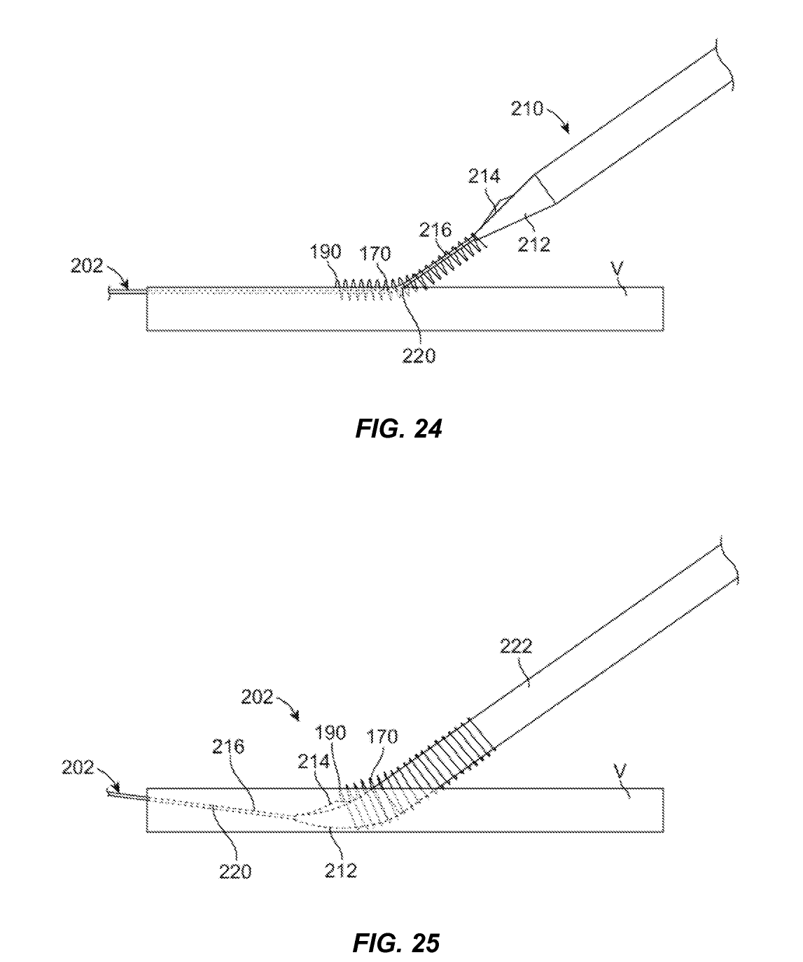

At this point, there are a number of options in the procedure. An interventional device may be introduced directly over the guidewire 202, through the tissue tract and into the lumen of the blood vessel V. This option is feasible when the interventional device has a smoothly tapered distal end that will pass through the vessel wall by gradually dilating the puncture site. The diameter of the interventional device would preferably be smaller than the diameter of the helical coil of suture 170 so that it could easily pass through the coil into the lumen of the blood vessel V. (Alternatively, a stretchable or extendable suture, as described herein below, would allow an interventional device that is actually larger in diameter than the helical coil of suture 170 to pass through.) An example of a device suitable for this variation of the method would be a large dilatation balloon, such as a valvuloplasty balloon. Another option is to insert an introducer sheath with a coaxial dilator over the guidewire 202, through the tissue tract and into the lumen of the blood vessel V. An introducer sheath allows interventional devices that might have a more a complex geometry with projections that might otherwise catch or snag on the suture 170 or the vessel wall to be easily passed through the puncture site into the lumen of the blood vessel V. An example of a device suitable for this variation of the method would be a stent graft for repair of abdominal aortic aneurysms. For interventional devices requiring a large diameter introducer sheath it may not be sufficient to simply dilate the puncture through the vessel wall because the vessel wall might tear rather than gradually dilate as intended. An example of a device that might require a large diameter introducer sheath might be a catheter for implanting a stented percutaneous aortic valve replacement. For this situation, the present invention includes, as an option, a cutting or scoring dilator 210 that is illustrated in FIGS. 24 and 25.

The cutting or scoring dilator 210 has a tapered dilating tip 212 on the distal end of a cylindrical body. A cutting or scoring element 214 located on one side of the tapered portion 212. The cutting or scoring element 214 is oriented longitudinally on the dilator 210 and is preferably located at a 12 o'clock position. A line or other mark on the proximal end of the dilator 210 indicates the orientation of the cutting or scoring element 214 to the operator. The cutting or scoring element 214 may be configured as a sharp cutting blade that actually cuts the vessel wall along a longitudinal line or it may be a wire, a wedge or a raised ridge that causes a stress riser in the vessel wall so that it preferentially splits or tears along a longitudinal line as the puncture site is dilated. Preferably, the cutting or scoring element 214 does not extend to the full outer diameter of the dilator 210, so that last bit of the insertion site through the vessel wall is dilated rather than cut or split. This provides better hemostasis at the insertion site and, in the case of a cutting or scoring element 214 configured as a sharp cutting blade, prevents the blade from cutting the helical coil of suture 170 that is in place. Alternatively or in addition, the cutting or scoring element 214 may have an electrocautery or electrocoagulation capability. Optionally, the cutting or scoring dilator 210 may also have a flexible lead section 216 that is smaller in diameter extending distally from the tapered dilating tip 212. The flexible lead section 216 improves the ability of the cutting or scoring dilator 210 to follow the guidewire 202 around the bend 206 into the lumen of the blood vessel V. A guidewire lumen 220 extends through the flexible lead section 216 and the body 218 of the cutting or scoring dilator 210. Alternatively, the cutting or scoring element 214 may be located on this flexible lead section 216. Preferably, a thin-walled introducer sheath 222 is positioned coaxially around the body 218 of the cutting or scoring dilator 210. Alternatively, a thin-walled introducer sheath 222 can be collapsed flat and introduced beside the body 218 of the cutting or scoring dilator 210. The introducer sheath 222 would be opened up to its full diameter after the dilator 210 has been withdrawn.

FIG. 24 shows the cutting or scoring dilator 210 following the guidewire 202 through the tissue tract. The distal tip of the flexible lead section 216 is positioned to enter the puncture site through the vessel wall. FIG. 25 shows the cutting or scoring dilator 210 with the tapered portion 212 inside the lumen of the blood vessel V. By a combination of cutting, tearing or splitting and dilating, the cutting or scoring dilator 210 has enlarged the puncture site to an insertion site large enough for the introducer sheath 222. The cutting or scoring dilator 210 also passes through the helical coil of suture 170 and may optionally dilate it to a larger diameter.

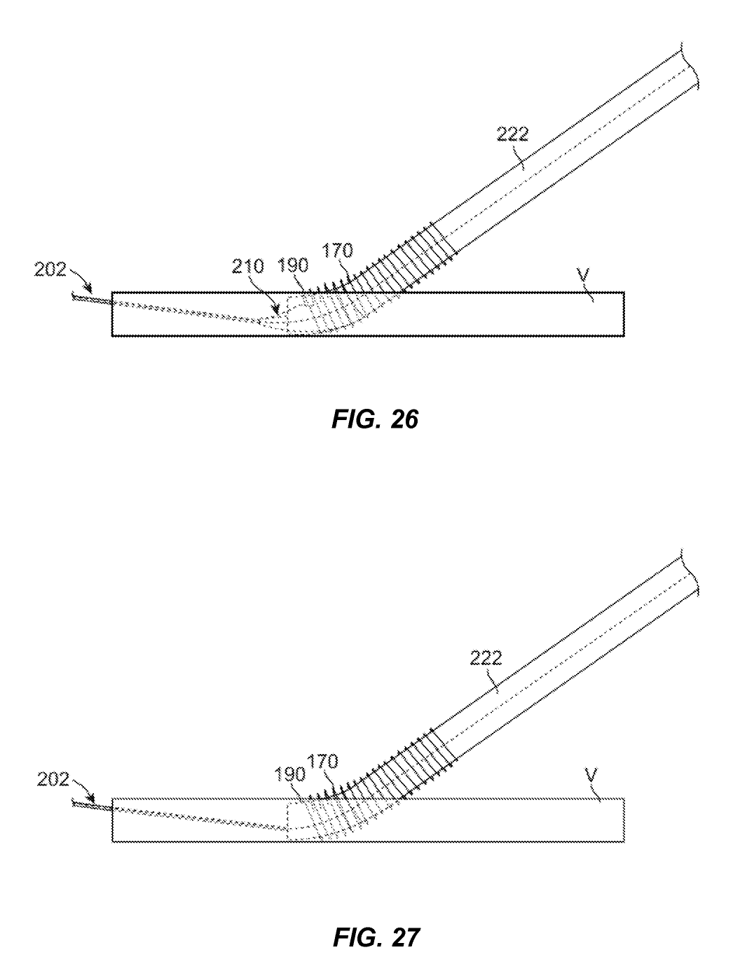

FIG. 26 shows the cutting or scoring dilator 210 being withdrawn, leaving the introducer sheath 222 in place through the tissue tract and into the lumen of the blood vessel V. The introducer sheath 222 also passes through the center of the helical coil of suture 170, as shown in FIG. 27.

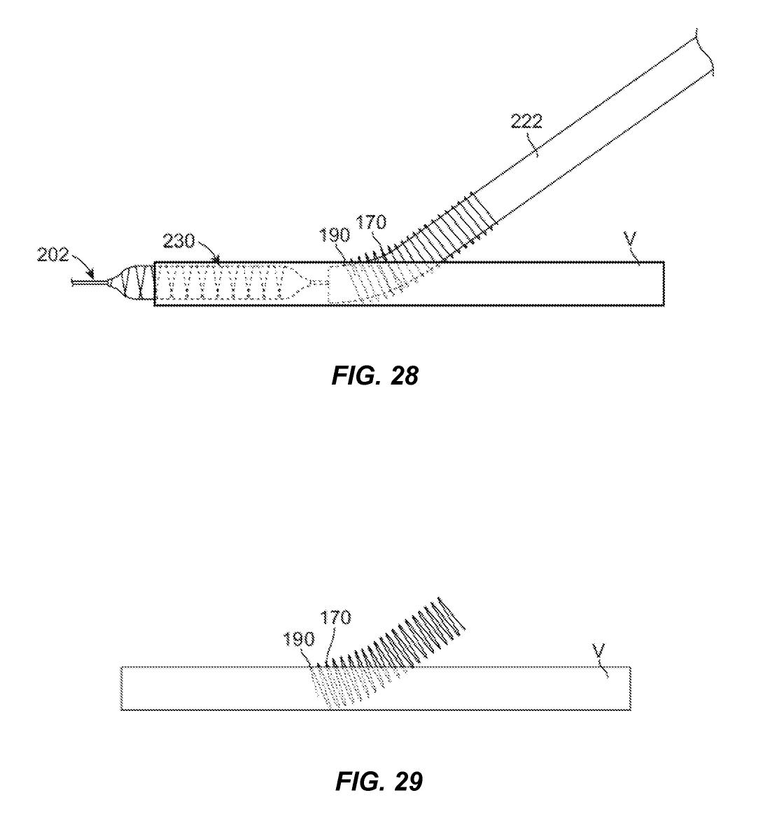

Once the introducer sheath 222 is in place, a variety of diagnostic, therapeutic and/or interventional devices 230 can be inserted through the introducer sheath 222, as shown in FIG. 28. The guidewire 202 may be used to introduce the interventional device 230 or it may be withdrawn and discarded if it is of no further use in the procedure. The interventional procedure may be performed anywhere in the vasculature that is accessible from the insertion site.

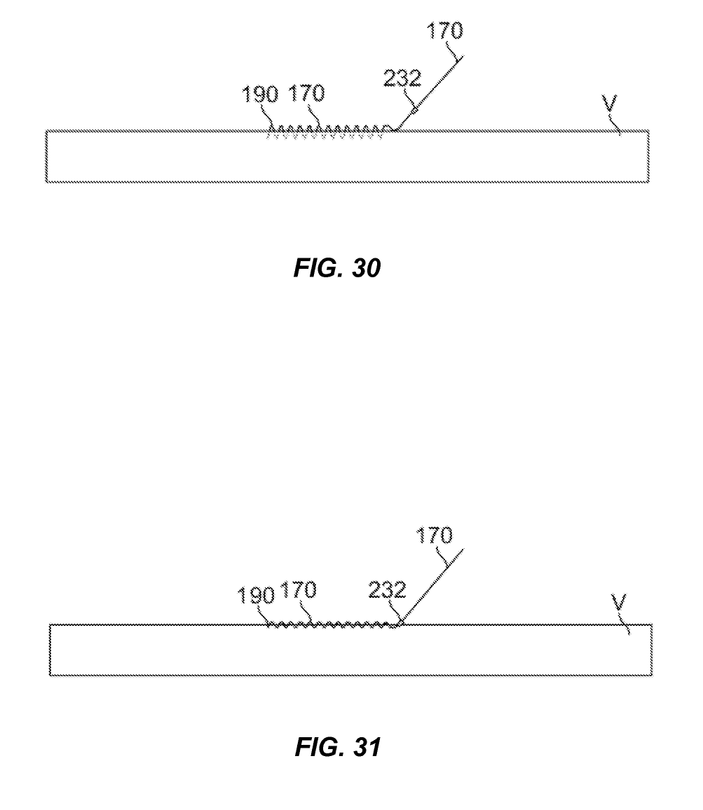

Once the interventional procedure has been completed, the interventional device 230 and then the introducer sheath 222 are withdrawn, leaving only the helical coil of suture 170 in place, as shown in FIG. 29. The suture 170 is pulled until it tightens from a loose coil into a running suture that closes the insertion site, as shown in FIG. 30. A knot or a suture lock 232 is placed on the suture 170 and slid down the suture 170 to lock the running suture in place, as shown in FIG. 31. A tube or a surgical knot pusher can be used to push the knot or suture lock 232 down through the tissue tract and along the suture 170. Optionally, the suture 170 may be cut off proximal to the suture lock 232. Optionally, an adhesive or sealant may be applied to the suture 170 and the insertion site. If necessary, additional sutures, adhesives or collagen plugs may be used to close and/or promote healing of the tissue tract.

A radiopaque contrast agent can be injected for confirmation of positioning and mapping of the blood vessel and its sidebranches by fluoroscopy at different points during the procedure. For example, the access needle 200, the guiding element 242, the vessel access and closure device 100, the dilator 210 and the introducer sheath 222 each have a lumen that can be used for radiopaque dye injections. In addition, each of the components may have radiopaque markers and/or be made of a radiopaque material to facilitate fluoroscopic imaging.

The following are given as nonlimiting examples of the dimensions and materials for some of the components of the vessel access and closure device 100. The helical suture needle 132 will preferably have a needle diameter in the range of approximately 0.015-0.050 inches, a helix diameter in the range of approximately 0.100-0.500 inches, and a length in the range of approximately 0.25-1.5 inches. The pitch or coil-to-coil distance of the helical suture needle 132 will preferably be in the range of approximately 0.030-0.125 inches and the number of coils or turns will be approximately 6-20. The elongated shaft portion 104 will preferably have an outside diameter in the range of approximately 0.100-0.375 inches and a length in the range of approximately 3-18 inches. The suture 170 will preferably be size 5-0 or larger and may be monofilament, braided, profiled shape (mono or braided), coated, dipped and/or lubricated and may be made from nylon, ultra high molecular weight polyethylene, silk, gut, expanded PTFE, absorbable polymers, etc. The guidewire will preferably have a diameter in the range of approximately 0.014-0.045 inches, more preferably 0.035-0.038 inches, though other sizes may also be used. The cutting or scoring dilator 210 will preferably have an outside diameter in the range of approximately 6-24 French (2-8 mm) and the introducer sheath 222 will preferably have an inside diameter in the range of approximately 6-24 French that is matched to the outside diameter of the cutting or scoring dilator 210.

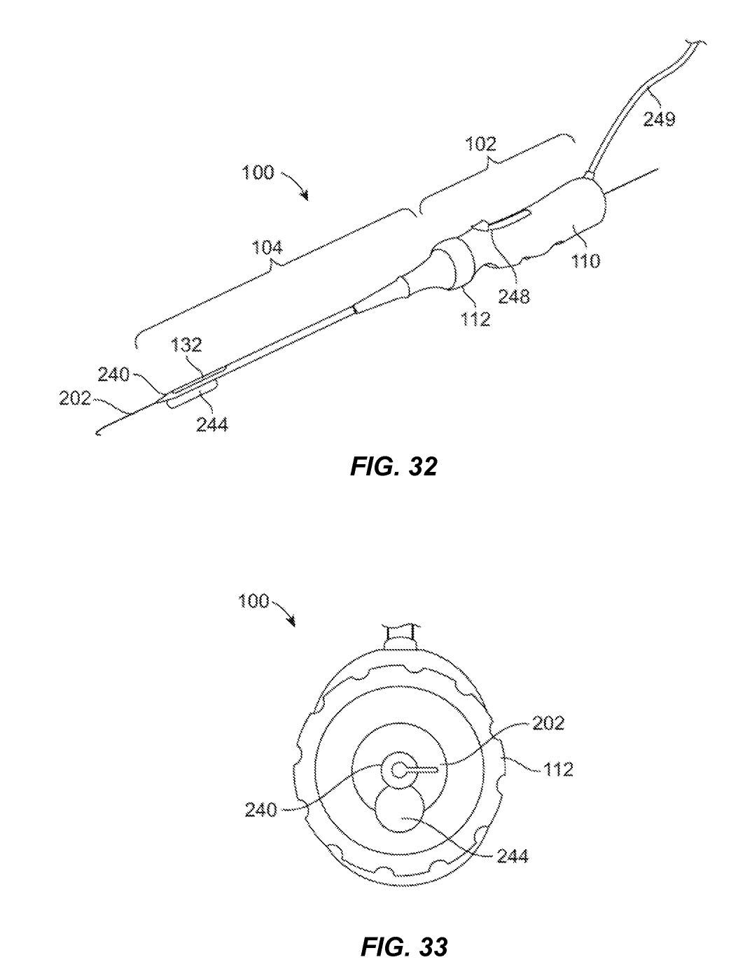

FIG. 32 is a perspective view and FIG. 24 is a front view illustrating another embodiment of the vessel access and closure device 100 incorporating some additional features. The vessel access and closure device 100 has an elongated shaft portion 104 connected to a proximal handle 102. In this embodiment, the rotating portion 112 is located on the distal end of the proximal handle 102, distal to the stationary portion 110. The rotating portion 112 is connected to the torque transmitting member 124 by a planetary gear mechanism or the like (not shown). A positioning device 240, similar to the one described above, is incorporated into the device 100. A sliding control button 248 on the proximal handle 102 controls the advancement and retraction of a retractable cutter that cuts a larger access opening at the puncture site. Optionally, the positioning device 240 may also be made retractable. Another sliding control button could be located on the proximal handle 102 to control the advancement and retraction of the positioning device 240. An inflation tube with a stopcock 249 connects to a pressure source, such as a syringe (not shown), for inflating and deflating the balloon 244. Because the positioning device 240 is connected to the proximal handle 102, the correct orientation of the balloon 244 in the blood vessel is assured.

In other embodiments of the vessel access and closure device 100, a motor or other mechanism may be provided to drive the rotation of the helical suture needle 132. The motor may be located in the proximal or distal end of the device 100. Other manually operated mechanisms may also be used to drive the rotation of the helical suture needle 132. For example, a handle or trigger may be connected to the torque transmitting member 124 by a rack-and-pinion or other gear mechanism that turns linear motion to rotary. The handle or trigger would be squeezed to rotate the helical suture needle 132. A lever or knob may be provided to reverse the direction of rotation.

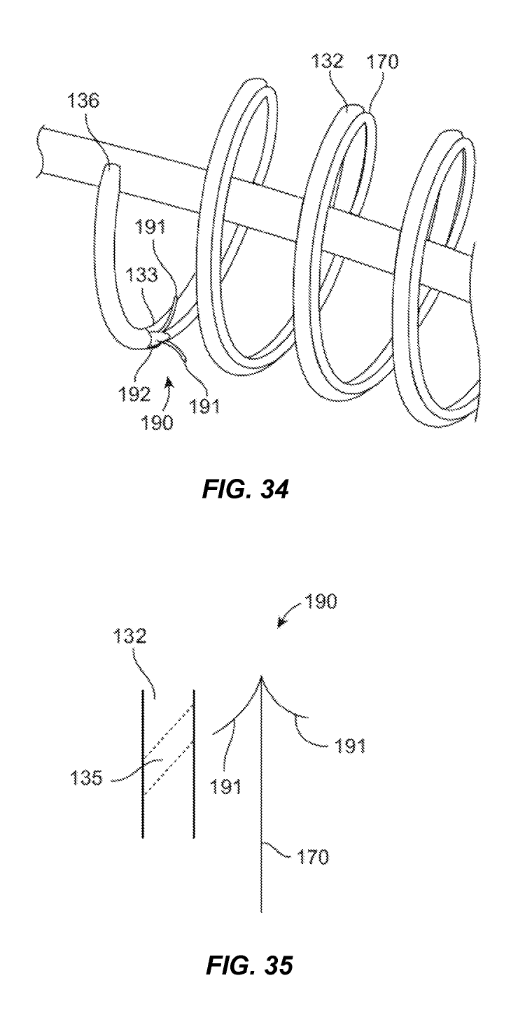

FIG. 34 shows an enlarged view of the helical suture needle 132 with the suture 170 and the suture anchor 190. The suture anchor 190 is attached to the distal end of the suture 170, for example by adhesive, overmolding, crimping, swaging, tying or forming integrally with it. The suture anchor 190 is releasably attached to the helical suture needle 132 by a ring or collar 192 that fits around the suture needle 132 and rests against a shelf or ledge 133 on the suture needle 132. The suture anchor 190 has at least one, and preferably two or more, resilient barbs 191 that are angled backward so the suture anchor 190 will move easily through the tissue in a forward direction along with the helical suture needle 132. When the direction of the helical suture needle 132 is reversed, the barbs 191 will spread to anchor the suture anchor 190 and the suture 170 to the blood vessel wall. The reverse motion will also dislodge the collar 192 of the suture anchor 190 from the shelf or ledge 133, thus releasing the suture anchor 190 from the suture needle 132.

FIG. 35 shows an enlarged view of another variation of the suture anchor 190. The suture anchor 190 is attached to the distal end of the suture 170, for example by adhesive, overmolding, crimping, swaging, tying or forming integrally with it. As above, the suture anchor 190 has a pair of resilient barbs 191 that are angled backward. In this variation, the suture anchor 190 is releasably attached to the helical suture needle 132 by inserting one of the barbs 191 into an obliquely drilled hole 135 in the suture needle 132. The backward-angled resilient barb 191 allows the suture anchor 190 to move easily through the tissue in a forward direction along with the helical suture needle 132. When the direction of the helical suture needle 132 is reversed, the barbs 191 will spread to anchor the suture anchor 190 and the suture 170 to the blood vessel wall. The reverse motion will also dislodge the suture anchor 190 from the hole 135, thus releasing the suture anchor 190 from the suture needle 132.

As mentioned previously, the helical suture needle 132 may be tubular, formed for example from stainless steel or NiTi alloy hypodermic needle tubing. The suture 170 and the suture anchor 190 may fit inside of the helical suture needle 132, as shown in FIG. 27. The suture anchor 190 may have barbs, as described above, or it may be configured as a simple toggle 193 attached near its middle to the suture 170. After the helical suture needle 132 has advanced through the blood vessel wall, the toggle 193 is ejected from the helical suture needle 132, preferably on the exterior of the blood vessel, to anchor the suture 170, as shown in FIG. 28.

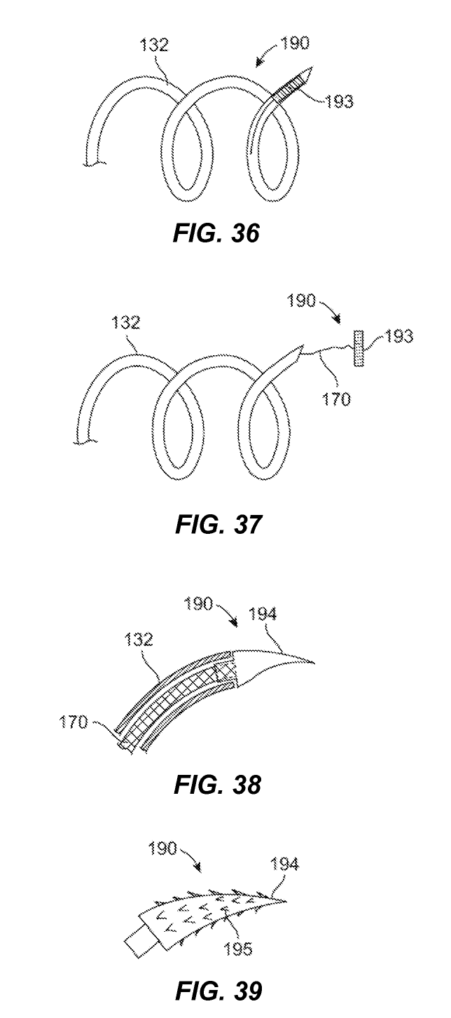

FIG. 36 shows a suture anchor 190 with a tissue-piercing point 194 that is configured to fit into the tubular distal end of a helical suture needle 132. The suture anchor 190 may have barbs, as described above, or it may be attached to the suture 170 near its middle to act as a toggle fastener.

FIG. 39 shows another suture anchor 190 with a tissue-piercing point 194 that is configured with a multiplicity of small barbs 195 to anchor the suture 170 to the blood vessel wall or surrounding tissue.

The suture anchors 190 shown in FIGS. 29 and 30 can also be adapted fit onto the distal end of a solid helical suture needle 132.

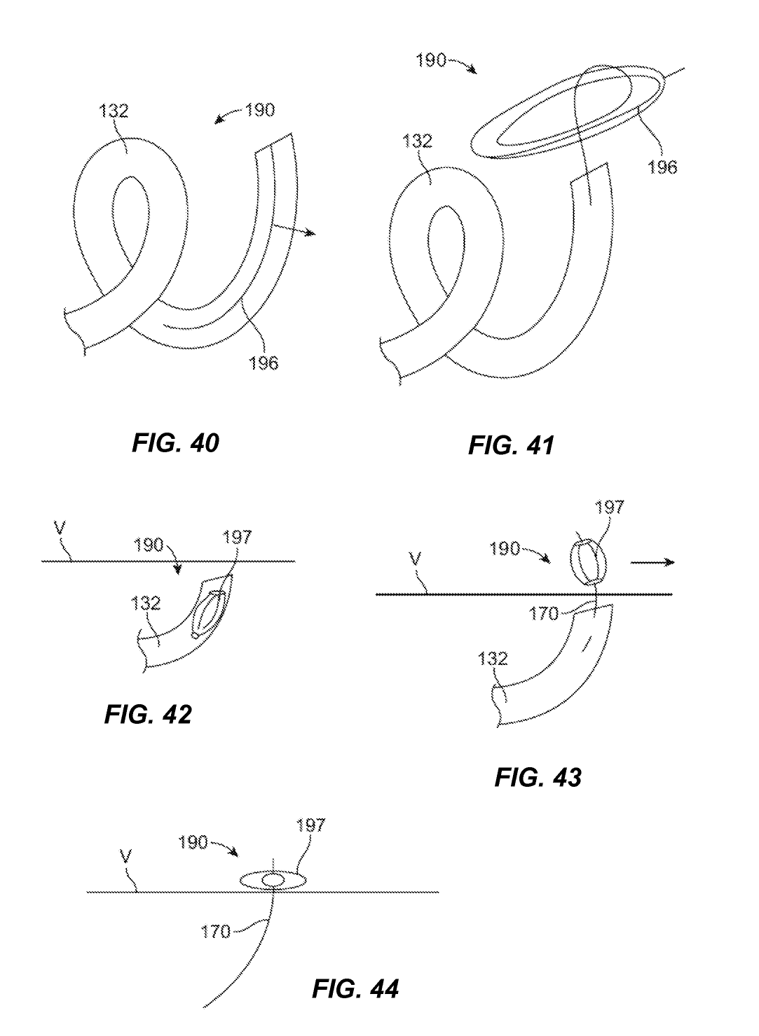

FIGS. 40 and 41 show a distal portion of a tubular helical suture needle 132 with a suture anchor 190 made of a superelastic or shape memory NiTi alloy wire 196. A distal portion of the wire 196 is preformed by heat treating into a curvature, for example a spiral coil, that will act as a suture anchor 190, as shown in FIG. 32. The curvature in the wire 196 can be straightened out by drawing it into tubular helical suture needle 132, as shown in FIG. 31. After the helical suture needle 132 has advanced through the blood vessel wall, the wire 196 is advanced out of the helical suture needle 132, preferably on the exterior of the blood vessel, and the curvature reforms to anchor the suture 170, as shown in FIG. 32.

FIGS. 42, 43, and 44 show a distal portion of a tubular helical suture needle 132 with a suture anchor 190 configured as an expandable cage 197, preferably of superelastic or shape memory NiTi alloy wire. The expandable cage 197 can be compressed to fit into the tubular helical suture needle 132, as shown in FIG. 33. After the helical suture needle 132 has advanced through the blood vessel wall, the expandable cage 197 is ejected from the helical suture needle 132, preferably on the exterior of the blood vessel, and the expandable cage 197 expands to anchor the suture 170, as shown in FIG. 43. FIG. 44 shows the expandable cage 197 of the suture anchor 190 anchoring the suture 170 to the wall of the blood vessel V.

The following describes additional features of the invention that may be combined with the embodiments of the vessel access and closure device 100 described above.

Optionally, excitation of the helical suture needle 132 with subsonic, sonic or ultrasonic vibration may be used to facilitate passing the needle through the wall of the blood vessel. This feature may be especially advantageous when the walls of the blood vessel are heavily calcified. Another way to facilitate passing the needle through the wall of the blood vessel would be to wind up and release stored spring energy in the helical suture needle 132 to move the distal tip 136 of the needle forward quickly to pierce the vessel wall.

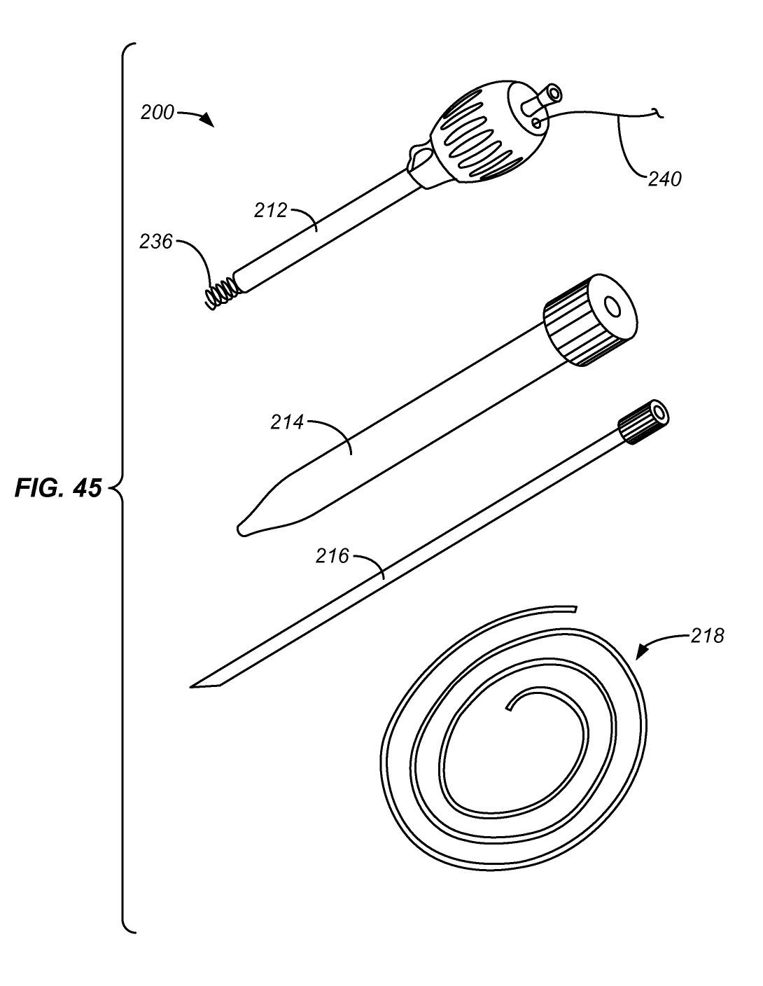

Referring to FIG. 45, a system 210 constructed in accordance with the principles of the present invention includes a helical needle driver 212, a dilator 214, a straight needle 216, and optionally a guidewire 218. The components of the system will typically be packaged together in conventional packaging, such as plastic trays, sterilized bags, boxes, and the like. The relative dimensions of each of the components will be selected to be compatible with each other. For example, both the helical needle driver 212 and dilator 214 will be sized to be advanced over either the needle 216 (in embodiments where the needle will be used as the guide for introducing these tools through the myocardium), or over the guidewire 218 (in embodiments where the driver 212 and dilator 214 will be advanced over the guidewire).

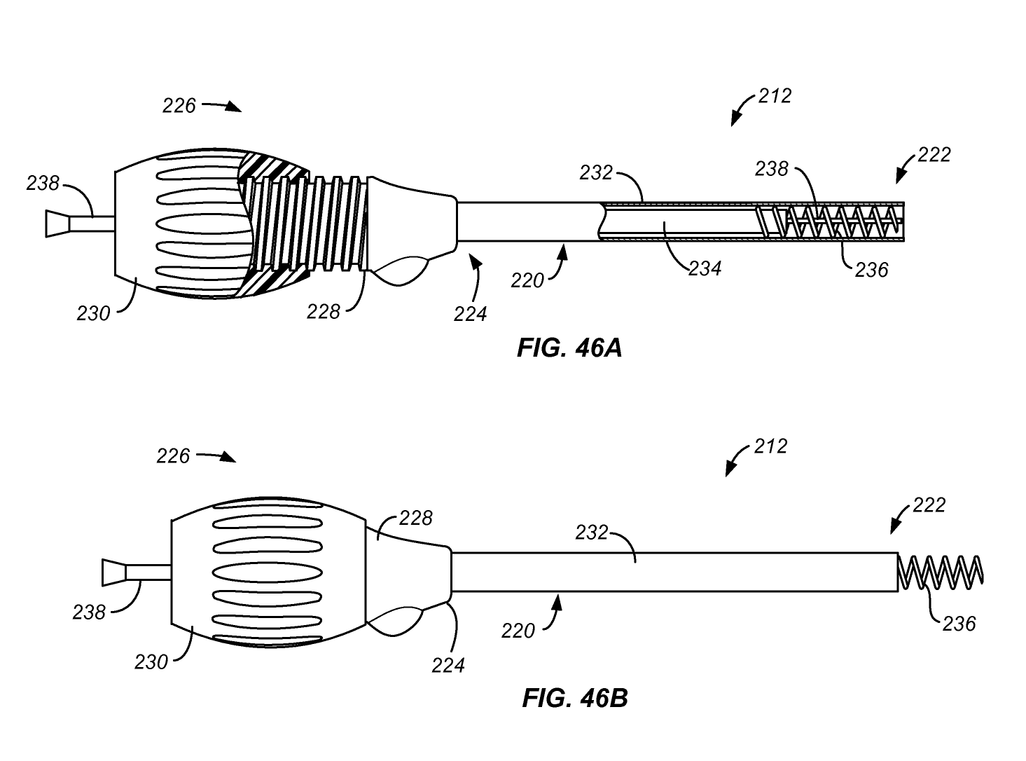

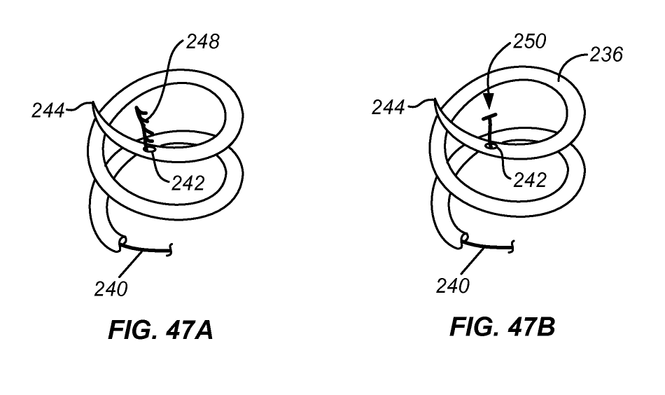

Referring now to FIGS. 46A and 46B, the helical needle driver 212 comprises a shaft assembly 220 having a distal end 222 and a proximal end 224. A drive handle 226 is attached to the proximal end 224 of the shaft assembly 220 and includes an inner threaded body 228 (FIG. 46A) and an outer rotatable member 320. The outer rotatable member 230 can be rotated over the inner threaded body 228 so that a helical needle 36 can be selectively retracted and advanced as shown in FIGS. 246 and 46B, respectively.

The inner threaded body 228 of the drive handle 226 is fixedly attached to an outer cylindrical tube 232 of the shaft assembly 220 while the outer rotatable member 230 is attached to an inner tubular member 234 (FIG. 46A). In this way, rotation of the outer rotatable member 230 over the inner threaded body 228 both rotates and advances (or retracts) the helical needle 236 which is fixedly attached to a distal end of the inner tubular member 234. Although shown as a simple helical needle, the needle in the helical needle driver can have any of the configurations.

The helical needle driver 212 also includes a central tube 238 which extends the entire length thereof and which provides a central passage way or lumen for advancement of the driver over the straight needle 216 and/or guidewire 218, as described in more detail below.

Referring to FIGS. 47A and 47B, suture 240 will typically be stowed or held within a hollow passageway through at least a distal portion of the needle 236. The suture will extend out of a small hole or port 242 disposed near the sharpened tip 244 of the needle. The suture will have an anchor formed at or over its exposed end. The anchor may be a barbed structure 248, as show in FIG. 47A, a T-Bar structure 50, as shown in FIG. 47B, or any one of a variety of other structures which allow the suture to be advanced into the tissue and which anchor within the tissue when the needle is counter-rotated and withdrawn from the tissue. The suture may be configured and/or deployed to accommodate expansion as the dilator is advanced through the helical “cage” formed after the suture is deployed. For example, the suture could be “stretchable” along its length so that the diameter of the helical cage can increase as the dilator is advanced. Alternatively, excess suture length can be stowed in and/or over the helical needle so that extra lengthing capacity is provided when the suture is left in the tissue.

What is claimed is:

1. A method for applying a controlled tension on tissue, said method comprising:
introducing a length of suture into a tissue bed through a tissue tract, wherein a distal end of the suture anchors in a distal region of the tissue tract; and
manually applying a pulling force in a proximal location on a proximal region of the suture length to apply proximal tension on the suture and anchor;
wherein the pulling force is applied through a coupling element which signals when the pulling force exceeds a target level.

2. A method as in claim 1, wherein a pulling force which exceeds the target level causes the coupling element to release the proximal region from the distal end of the suture.

3. A method as in claim 2, wherein the release is complete.

4. A method as in claim 2, wherein the release is partial.

5. A method as in claim 2, wherein the coupling element comprises a sleeve attached over the proximal region of the suture, wherein the sleeve is initially attached to the suture and detaches from the suture when the pulling force exceeds the target level.

6. A method as in claim 2, wherein the coupling element comprises a force measurement device which alerts the user when the pulling force exceeds the target level.

7. A method as in claim 1, wherein introducing the suture length comprises advancing a needle through a tissue bed to form a tissue tract and withdrawing the needle from the tissue tract after the tract has been formed, wherein the suture is carried by the needle and the anchor self-deploys in the tissue as the direction of the needle advancement reverses.

8. A method as in claim 7, wherein the anchor comprises bars which are swept back so that they allow the needle and suture to be advanced through tissue but which deploy into the tissue when the needle direction is reversed.

9. A method as in claim 8, wherein the barbs are exposed from the needle as the needle is advanced.

10. A suture construct comprising:
a length of suture having a distal end and a proximal region;
a tissue anchor attached to the suture length near its distal end; and
a coupling element which transmits a manual pulling force to the distal end of the suture and which signals when the pulling force exceeds a target level.

11. A suture construct as in claim 10, wherein the tissue anchor comprises barbs over at least the distal end of the suture, wherein the barbs are swept back in a proximal direction to allow the suture to be advanced distally through suture but prevent the suture from being pulled proximally through tissue.

12. A suture construct as in claim 11, wherein the barbs are present only over the distal tip of the suture.

13. A suture construct as in claim 10, wherein the coupling element comprises a breakable link disposed between the distal end and the proximal region.

14. A suture construct as in claim 10, wherein the coupling element comprises an extendable loop disposed between the distal end and the proximal region.

15. A suture construct as in claim 10, wherein the coupling element comprises a break-away sleeve over the proximal region of the suture, wherein the sleeve allows manual grasping by the user and separates from the suture when the pulling force exceeds the target level.

16. A suture construct as in claim 10, wherein the coupling element comprises a force gauge which provides an indication or alarm when the pulling force exceeds the target level.

17. A method for anchoring a distal end of a length of suture in a tissue tract in a tissue bed, said method comprising:
providing a needle having a tissue-penetrating distal tip with the length of suture releasably secured over or through at least a distal portion of the needle;
advancing the needle into the tissue bed so that the suture follows the tract formed in the tissue bed by the needle; and
retracting the needle through the needle tract;
wherein a distal anchor on the distal end of the suture self-deploys in the tissue bed so that they suture separates from the needle and remains in the needle tract after the needle is withdrawn.

18. A method as in claim 17, wherein the needle is straight and forms a straight tissue tract when advanced in the tissue bed.

19. A method as in claim 17, wherein the needle is carved and forms a carved tissue tract when advanced into tissue.

20. A method as in claim 17, wherein the needle is helical and forms a helical tissue tract when advanced into tissue.

21. A method as in claim 17, wherein the anchor comprises a plurality of swept back barbs over at least a distal portion of the suture, wherein the barbs remain swept back while the needle and suture are advanced and the barbs deploy outwardly when the needle is pulled back through the tissue tract.

22. A method as in claim 21, wherein the barbs are not constrained while being advanced through the tissue bed.

23. A method as in claim 22, wherein the barbs are radially constrained while being advanced through the tissue bed and wherein the barbs are released from constraint immediately before withdrawing the needle from the tissue tract.

24. A method as in claim 17, wherein a proximal anchor on the proximal region of the suture self-deploys in the tissue tract to inhibit the proximal end of the suture from moving distally.

25. A method as in claim 24, wherein the proximal anchors comprises barbs which are swept distally to inhibit distal movement on deployment.

26. A method as in claim 25, wherein the proximal barbs are constrained during advancement of the needle and released after the distal anchor has been deployed.

27. A method as in claim 26, wherein release comprises release form the needle.

28. A method as in claim 26, wherein release comprises resorption of a resorbable restraing.

29. A method as in claim 26, wherein release comprises dissolving of a dissolvable restraing.
